(19) United States
(12) Patent Application Publication
(10) Pub. No.: US 2026/0171186 A1
(43) Pub. Date: Jun. 18, 2026

(54) SYSTEMS AND METHODS FOR MODELING THROMBIN-ANTITHROMBIN

(71) Applicant: Genzyme Corporation, Cambridge, MA (US)

(72) Inventors: Chanchala Kaddi, Framingham, MA (US); Randolph Leiser, Piscataway, NJ (US); Mengdi Tao, Gillette, NJ (US); Susana Zaph

(21) Appl. No.: 19/126,937

(22) PCT Filed: Oct. 31, 2023
(86) PCT No.: PCT/US2023/036417

§ 371 Date: May 2, 2025

Related U.S. Application Data
(60) Provisional application No. 63/421,912, filed on Nov. 2, 2022.
(60) Provisional application No. 63/431,186, filed on Dec. 8, 2022.

(51) Int. Cl. G16B 5/00; A61K 38/37; C12N 15/113

(52) U.S. Cl. CPC G16B 5/00; A61K 38/37; C12N 15/113; C12N 2310/14

(57) ABSTRACT

The present disclosure provides methods and systems for modeling, simulating, and treating hemophilia. The methods and systems disclosed herein include a quantitative systems pharmacology (QSP) model to predict features of thrombin generation in people with hemophila A and people with hemophilia B in the context of antithrombin (AT) lowering.

### CROSS-REFERENCE TO RELATED APPLICATIONS

This application claims the benefit of U.S. Provisional Patent Application No. 63/421,912, filed on Nov. 2, 2022 and U.S. Provisional Patent Application No. 63/431,186, filed Dec. 8, 2022, the contents of which are incorporated herein by reference in their entirety.

### TECHNICAL FIELD

The present disclosure relates to quantitative systems pharmacology (QSP) models of hemostatic dynamics in human subjects.

### BACKGROUND

In conditions of low AT, predicting thrombin activity and Factor VIII equivalency, particularly the contribution of reactions involving α-2-macroglobulin, remains challenging. There is a need to accurately predict thrombin activity in low AT conditions, accounting for the contribution of α-2-macroglobulin in a human body.

### SUMMARY

The present disclosure provides a quantitative systems pharmacology (QSP) model to predict or validate features of thrombin generation in PwHA and PwHB in the context of AT lowering. To better understand the hemostatic equivalency of fitusiran prophylaxis (i.e., AT lowering) a QSP model was developed to integrate clinical and nonclinical data of the coagulation pathway to predict the results of multiple in vitro coagulation assays, including the thrombin generation assay (TGA), and to mechanistically understand the readouts of these assays.

The QSP model represents reported steady-state levels of coagulation factors and accessory proteins in the plasma of healthy individuals and PwHA and PwHB. In addition, the model describes the impact of α-2-macroglobulin, a regulator of thrombin, with increased influence at reduced AT levels to predict the impact of fitusiran on thrombin generation and evaluate factor equivalency.

By considering the impact of α-2-macroglobulin, the QSP model provides a mechanistic representation of thrombin generation under conditions of AT lowering, consistent with TGA data from donor-derived spiked plasma and clinical TGA data from both PwHA and PwHB. The VP analysis of fitusiran prophylaxis provided hemostatic equivalency with FVIII in a representative population of severe PwHA, with a targeted therapeutic range of AT of 15-35% resulting in a thrombin peak profile which was comparable to 10-20 IU/kg FVIII. Future applications of the QSP model can include assessments of other therapeutics for PwHA and PwHB as well as extension to describe additional coagulation tests.

In one aspect, the disclosure provides a method of preparing a quantitative systems pharmacology model for predicting thrombin levels in plasma of a subject, the method comprising one or more of the following steps:
(a) providing a plurality of relationships and/or parameters characterizing time-dependent antithrombin (AT) levels in plasma of a subject in response to an agent targeting antithrombin in the plasma of the subject;
(b) providing a plurality of relationships and/or parameters characterizing α-2-macroglobulin levels in the plasma of the subject;
(c) determining a plurality of rate constants for the plurality of relationships and/or parameters characterizing time-dependent AT levels in plasma of a subject in response to an agent targeting antithrombin in the plasma of the subject;
(d) determining a rate constant for the relationship or the parameter characterizing α-2-macroglobulin levels in the plasma of the subject; and/or
(e) programming a computational system with (i) the plurality of rate constants for the plurality of relationships and/or parameters characterizing time-dependent AT levels in plasma of a subject in response to an agent targeting antithrombin in the plasma of the subject; and (ii) the rate constant for the relationship or the parameter characterizing α-2-macroglobulin levels in the plasma of the subject, whereby the computational system is programmed to (i) solve a system of expressions under a defined set of pharmacological conditions, wherein the system of expressions comprises the plurality of relationships and/or parameters characterizing time-dependent AT levels in plasma of a subject in response to an agent targeting antithrombin in the plasma of the subject and the plurality of relationships and/or parameters characterizing α-2-macroglobulin levels in the plasma of the subject and (ii) output thrombin levels in plasma of a subject after administration of the agent targeting antithrombin in the plasma of the subject under the defined set of pharmacological conditions.

In some embodiments, the agent targeting antithrombin in the plasma of the subject is an siRNA therapeutic. In some embodiments, the siRNA therapeutic is fitusiran.

In some embodiments, the AT levels are below 35%. In some embodiments, the method calculates the formation and the degradation of α-2-macroglobulin-thrombin complex. In some embodiments, α-2-macroglobulin has a concentration of about 3 to about 6 μM.

In one aspect, the disclosure provides a computer-implemented method for modeling and simulating thrombin levels in the plasma of a subject, the method comprising one or more of the following steps:
obtaining a quantitative systems pharmacology (QSP) model of thrombin levels in the plasma of a subject, wherein the QSP model is configured to represent results of a thrombin generation assay (TGA) in response to plasma levels of antithrombin (AT), α-2-macroglobulin, and a pharmaceutical agent targeting antithrombin in the plasma of the subject;
determining parameters affecting peak thrombin as indicated by the TGA;
assigning the parameters affecting peak thrombin to a virtual patient population; and/or
processing the virtual patient population using the QSP model to provide processed data, wherein the processed data comprises an amount of the agent targeting antithrombin in the plasma of the subject.

In some embodiments, the AT levels are below 35%.

In some embodiments, the method calculates the formation and the degradation of α-2-macroglobulin-thrombin complex.

In some embodiments, the method further involves displaying the processed data.

In some embodiments, the method further involves determining pharmacokinetic parameters of the agent targeting antithrombin in the plasma of the subject; determining pharmacokinetic parameters of one or more additional therapeutics; and/or processing the pharmacokinetic parameters of the agent targeting antithrombin in the plasma of the subject and the pharmacokinetic parameters of the one or more additional therapeutics to determine effectiveness of the combination of the agent targeting antithrombin in the plasma of the subject and one or more additional therapeutics.

In some embodiments, the agent targeting antithrombin in the plasma of the subject is an RNAi therapeutic. In some embodiments, the RNAi therapeutic is an siRNA therapeutic. In some embodiments, the siRNA therapeutic is fitusiran.

In one aspect, the disclosure provides a computer-implemented method for determining Factor VIII equivalency in a subject, the method comprising one or more of the following steps: obtaining a quantitative systems pharmacology (QSP) model of thrombin levels in the plasma of a subject, wherein the QSP model is configured to represent the interaction of plasma levels of antithrombin (AT), α-2-macroglobulin, and thrombin; determining parameters affecting a thrombin generation assay; and/or processing the QSP model to provide processed data, wherein the processed data indicates Factor VIII equivalency in a subject.

In some embodiments, the plasma levels of antithrombin is a time-dependent variable. In some embodiments, the plasma levels of antithrombin are below 35%.

In some embodiments, the formation and the degradation of thrombin-antithrombin (T-AT) are calculated.

In some embodiments, the formation and the degradation of α-2-macroglobulin-thrombin complex are calculated.

In some embodiments, the results of a thrombin generation assay (TGA) in response to the interaction of plasma levels of antithrombin (AT), α-2-macroglobulin, and thrombin are modeled.

In some embodiments, peak thrombin as indicated by the TGA is used to determine Factor VIII equivalency. In some embodiments, the subject is administered an siRNA therapeutic. In some embodiments, the siRNA therapeutic is fitusiran.

In one aspect, the disclosure provides a method of achieving a Factor VIII equivalency of about 10% to about 50% in a hemophilia patient in need thereof, the method comprising administering a prophylactically effective amount of fitusiran subcutaneously to the patient to achieve an AT level of 10-35% in the patient.

In some embodiments, the patient is a hemophilia A or hemophilia B patient. In some embodiments, the patient is a hemophilia A patient with or without inhibitors, or a hemophilia B patient with or without inhibitors.

In some embodiments, the method comprises achieving a Factor VIII equivalency of about 20% to about 40%.

In some embodiments, the prophylactically effective amount of fitusiran is selected from about 1.25 mg, about 2.5 mg, about 5 mg, about 25 mg, about 30 mg, about 50 mg, or about 80 mg.

In some embodiments, the prophylactically effective amount of fitusiran is administered about every month (or about every four weeks) or about once every two months (or about every eight weeks).

In some embodiments, the prophylactically effective amount of fitusiran is about 50 mg administered about every month (or about every four weeks) or about once every two months (or about every eight weeks).

In some embodiments, the prophylactically effective amount of fitusiran is about 20 mg administered about every month (or about every four weeks) or about once every two months (or about every eight weeks).

In one aspect, the disclosure provides a method of treating hemophilia in a patient with or without inhibitors, the method comprising one or more of the following steps:
(a) providing a time-dependent antithrombin level in plasma of the patient;
(b) providing a plurality of relationships and/or parameters characterizing α-2-macroglobulin level in the plasma of the patient;
(c) programming a computational system with one or more rate constants for the relationship or the parameters characterizing thrombin level;
(d) determining hemostatic equivalency with Factor VIII in the patient; and/or
(e) administering to the patient a therapeutic agent.

In some embodiments, the therapeutic agent is Supplemental Factor VIII. In some embodiments, the therapeutic agent is fitusiran.

In some embodiments, hemostatic equivalency with Factor VIII is determined by modeling the interaction of plasma levels of antithrombin (AT), α-2-macroglobulin, and thrombin. In some embodiments, the time-dependent antithrombin level is below 35%.

In one aspect, the disclosure provides a method of determining hemostatic equivalency with Factor VIII in a subject receiving treatment for hemophilia A or B, the method comprising one or more of the following steps:

(a) providing a time-dependent antithrombin level in plasma of the subject;

(b) providing a plurality of relationships and/or parameters characterizing α-2-macroglobulin level in the plasma of the subject;

(c) programming a computational system with one or more rate constants for the relationship or the parameters characterizing thrombin level; and/or

(d) determining hemostatic equivalency with Factor VIII in the subject.

In some embodiments, the method further involves administering supplemental Factor VIII to the subject. In some embodiments, the method further involves adjusting the dosage of fitusiran for the subject.

In one aspect, the disclosure provides a method of achieving a Factor VIII equivalency of about 10% to about 50% in a hemophilia patient in need thereof, the method comprising administering a prophylactically effective amount of fitusiran subcutaneously to the patient to achieve a Factor VIII equivalency of about 10% to about 50% in the patient. In some embodiments, the method comprises achieving a Factor VIII equivalency of about 20% to about 40%.

In some embodiments, the model does not consider the PK/PD of fitusiran. Instead, a time-dependent AT level is provided.

As used herein, the term “about” generally means within 10%, 5%, 1%, or 0.5% of a given value or range. Alternatively, the term “about” means within an acceptable standard error of the mean when considered by one of ordinary skill in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a flowchart for an exemplary method of generating a QSP model of the present disclosure.

FIG. 2 is a flowchart for an exemplary method of using a QSP model of the present disclosure.

FIG. 3 is a process diagram representing species and reactions involved in the modeling and simulating of hemostasis in the context of treatment with fitusiran in accordance with some embodiments of the present disclosure.

FIG. 4 is a biological process map representing the roles of antithrombin, thrombin, and α-2-macroglobulin in accordance with some embodiments of the present disclosure.

FIG. 5A is a plot of thrombin concentration in plasma (nM) over time, comparing thrombin levels predicted by the QSP model disclosed herein (black line) to previously collected clinical data, with 100% antithrombin levels and 100% supplemental Factor VIII (Advate) levels.

FIG. 5B is a plot of thrombin concentration in plasma (nM) over time, comparing thrombin levels predicted by the QSP model disclosed herein (black line) to previously collected clinical data, with 20% antithrombin levels and 50% supplemental Factor VIII (Advate) levels.

FIG. 5C is a plot of α2M-IIa complex concentration (y-axis) against supplemental Factor VIII (Advate) levels (x-axis), comparing α2M-Ila complex levels predicted by the QSP model disclosed herein (black datapoints) to previously collected clinical data, with 100% antithrombin levels.

FIG. 5D is a plot of α2M-IIa complex concentration (y-axis) against supplemental Factor VIII (Advate) levels (x-axis), comparing α2M-IIa complex levels predicted by the QSP model disclosed herein (black datapoints) to previously collected clinical data, with 20% antithrombin levels.

FIG. 5E is a plot of α2M-IIa complex concentration (y-axis) against supplemental Factor VIII (Advate) levels (x-axis), comparing α2M-IIa complex levels predicted by the QSP model disclosed herein (black datapoints) to previously collected clinical data, with 10% antithrombin levels.

FIG. 5F is a plot of α2M-IIa complex concentration (y-axis) against supplemental Factor VIII (Advate) levels (x-axis), comparing α2M-IIa complex levels predicted by the QSP model disclosed herein (black datapoints) to previously collected clinical data, with 5% antithrombin levels.

FIG. 5G is a plot of thrombin concentration in plasma (nM) over time, comparing thrombin levels predicted by a previous model of the coagulation cascade that did not include α-2-macroglobulin (black line) to previously collected clinical data, with 100% antithrombin levels and 100% supplemental Factor VIII (Advate) levels.

FIG. 5H is a plot of thrombin concentration in plasma (nM) over time, comparing thrombin levels predicted by a previous model of the coagulation cascade that did not include α-2-macroglobulin (black line) to previously collected clinical data, with 20% antithrombin levels and 50% supplemental Factor VIII (Advate) levels.

FIG. 6 is a plot of peak thrombin levels (nM) for patients with a range of HEMA severity (residual factor VIII levels) observed in clinical data at different AT % ranges (x-axis).

FIG. 7A is a plot representing aggregate predictive performance of the QSP model disclosed herein, plotting predicted peak thrombin concentration (y-axis) over AT % (x-axis).

FIG. 7B is a plot representing aggregate predictive performance of the QSP model disclosed herein, plotting peak thrombin concentration observed in clinical trial (y-axis) over peak thrombin concentration predicted by the QSP model disclosed herein (x-axis). Dotted line represents correlation of observed and predicated peak thrombin concentration.

FIG. 8 is a box plot of peak thrombin concentration (nM) predicted by the QSP model disclosed herein for 100% AT (1), ˜10-20% AT (2), ˜10-25% AT (3), and ˜20-25% AT (4) for three dosages of supplemental Factor VIII. Predicted peak thrombin concentrations correspond to TGA assays performed on AT- and Factor VIII-double depleted plasma that was spiked with different levels of AT and/or Factor VIII.

FIG. 9A is a heatmap of peak thrombin (nM) distribution plotted against antithrombin % (y-axis) and supplemental Factor VIII dosage (x-axis). Quadrants of the plot are indicated, which correspond to FIGS. 9B, 9C, 9D, and 9E.

FIG. 9B is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 5% antithrombin levels and 0% supplemental Factor VIII (Advate) levels. FIG. 9B corresponds to the “1” indication on the heatmap of FIG. 9A.

FIG. 9C is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 5% antithrombin levels and 100% supplemental Factor VIII (Advate) levels. FIG. 9C corresponds to the “2” indication on the heatmap of FIG. 9A.

FIG. 9D is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 100% antithrombin levels and 0% supplemental Factor VIII (Advate) levels. FIG. 9D corresponds to the “3” indication on the heatmap of FIG. 9A.

FIG. 9E is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 100% antithrombin levels and 100% supplemental Factor VIII (Advate) levels. FIG. 9E corresponds to the “4” indication on the heatmap of FIG. 9A.

FIG. 10A is a heatmap of peak thrombin (nM) distribution plotted against antithrombin % (y-axis) and supplemental Factor VIII dosage (x-axis). Labels “1,” “2,” “3,” and “4,” are indicated, which correspond to FIGS. 10B, 10C, 10D, and 10E.

FIG. 10B is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 20% antithrombin levels and 5% supplemental Factor VIII (Advate) levels. FIG. 10B corresponds to the “1” indication on the heatmap of FIG. 10A.

FIG. 10C is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 24% antithrombin levels and 50% supplemental Factor VIII (Advate) levels. FIG. 10C corresponds to the “2” indication on the heatmap of FIG. 10A.

FIG. 10D is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 40% antithrombin levels and 20% supplemental Factor VIII (Advate) levels. FIG. 10D corresponds to the “4” indication on the heatmap of FIG. 10A.

FIG. 10E is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 30% antithrombin levels and 50% supplemental Factor VIII (Advate) levels. FIG. 10E corresponds to the “3” indication on the heatmap of FIG. 10A.

FIG. 11A is a heatmap of peak thrombin (nM) distribution plotted against antithrombin % (y-axis) and supplemental Factor VIII dosage (x-axis). Labels “5,” “6,” “7,” and “8,” are indicated, which correspond to FIGS. 11B, 11C, 11D, and 11E.

FIG. 11B is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 12% antithrombin levels and 100% supplemental Factor VIII (Advate) levels. FIG. 11B corresponds to the “5” indication on the heatmap of FIG. 11A.

FIG. 11C is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 100% antithrombin levels and 50% supplemental Factor VIII (Advate) levels. FIG. 11C corresponds to the “6” indication on the heatmap of FIG. 11A.

FIG. 11D is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 34% antithrombin levels and 50% supplemental Factor VIII (Advate) levels. FIG. 11D corresponds to the “7” indication on the heatmap of FIG. 11A.

FIG. 11E is a plot of simulated thrombin concentration in plasma (nM) over time, comparing thrombin levels predicted by the QSP model disclosed herein (black line) to AT- and Factor VIII-double depleted plasma that was spiked with different levels of AT and/or Factor VIII, with 12% antithrombin levels and 5% supplemental Factor VIII (Advate) levels. FIG. 11E corresponds to the “8” indication on the heatmap of FIG. 11A.

FIG. 12 is a plot of simulated activated partial thromboplastin time (aPTT) in plasma (seconds), comparing aPTT predicted by the QSP model disclosed herein to previously collected clinical data and data from the literature, for healthy subjects (left), hemophilia A subjects (center), and warfarin-treated subjects (right).

FIG. 13A is a plot of simulated activated partial thromboplastin time (aPTT) in plasma (seconds), plotted against supplemental Factor VIII % (x-axis) for simulated patients with 0.1% residual Factor VIII. 5%, 10%, 20%, and 100% AT are plotted.

FIG. 13B is a plot of data for activated partial thromboplastin time (aPTT) in plasma (seconds), plotted against spiked-in supplemental Factor VIII % (x-axis) for clinical plasma samples. 5%, 10%, 20%, and 100% AT are plotted.

FIG. 14 is a plot of peak thrombin (nM) data from the literature, for patients with hemophilia A (circles) and healthy volunteers (triangles). X-axis from left to right indicate data for control with 50% residual AT; control with 10% residual AT; 50% residual AT; 10% residual AT; and health volunteers.

FIG. 15 is a table of peak thrombin (mean±standard deviation) for a simulated virtual population (n=1000) of severe Hemophilia A (0.1% residual Factor VIII).

FIG. 16 is a schematic of an exemplary computer system used to implement the QSP models of the present disclosure.

FIG. 17 shows the expanded structural formula, chemical formula, and molecular mass of fitusiran.

FIG. 18 is a plot of predicted peak thrombin (nM) based on QSP model simulations for therapeutic antithrombin levels of 15-35% with fitusiran and FVIII activity of 20-40% in people with hemophilia A.

### DETAILED DESCRIPTION

Aspects of the present disclosure provide methods for modeling hemostatic equivalency of fitusiran prophylaxis, particularly relating to thrombin generation in PwHA and PwHB in the context of antithrombin (AT) lowering. In some embodiments, the QSP model simulates the association and dissociation of AT and thrombin, of α-2-macroglobulin and thrombin, and of thrombin and fibrinogen.

Use of the QSP model described in the present application can provide various types of information regarding fitusiran, thrombin, antithrombin, and α-2-macroglobulin interactions which may be impractical or impossible to clinically obtain. For example, the QSP model can provide, as an input, simulations of in vivo conditions of a subject. The in vivo conditions can include, but are not limited to, fitusiran plasma pharmacokinetics (PK), fitusiran liver PK, AT synthesis in liver, AT synthesis in plasma, synthesis of coagulation factors and coagulation proteins in plasma, elimination of coagulation factors and coagulation proteins in plasma. The QSP model can provide a simulated plasma sample of a subject. The simulated plasma sample can include parameters for AT concentration, factor II concentration, factor V concentration, factor VII concentration, factor VIII concentration, factor IX concentration, factor X concentration, factor XI concentration, factor XII concentration, α-2-macroglobulin concentration, Protein S concentration, Protein C concentration, thrombomodulin concentration, and prekallikrein concentration. The QSP model as described herein can perform a simulated thrombin generation assay (TGA) based on the simulated plasma sample of the subject. The QSP model can perform a simulated activated partial thromboplastin time (aPTT) test on the simulated plasma sample of the subject. The QSP model can provide, as an output, predicted peak height in a thrombin generation assay, area under the curve (AUC) in a thrombin generation assay, and lag time in a thrombin generation assay in samples from subjects administered fitusiran and/or supplemental Factor VIII.

The QSP model input can further include a particular dose and/or dose regimen of a therapeutic intervention for treating PwHA and/or PxHB, for example fitusiran with or without supplemental clotting factors. Accordingly, the QSP model can provide a quantitative relationship between a therapeutic intervention (e.g., a dosage or dose regimen of fitusiran with or without supplemental clotting factors) and a biomarker that provides a useful clinical target in treating patients, for example, peak thrombin and/or AT lowering. This quantitative relationship can be used to assist in determining in-human dosages of therapeutic interventions for HEMA and/or HEMB.

In some embodiments, the QSP model can be used for evaluating the efficacy of a therapeutic intervention for patients with a deficiency in the function of a clotting factor. In some embodiments, the clotting factor is Factor VIII or Factor IX. In some embodiments, the therapeutic intervention comprises administration of an RNAi therapeutic. In some embodiments, the RNAi therapeutic is a siRNA therapeutic. In some embodiments, the RNAi therapeutic is fitusiran. In some embodiments, the therapeutic intervention targets antithrombin (AT) to enhance thrombin generation (TG) and rebalance hemostasis in patients with HEMA or HEMA with or without inhibitors. The QSP model can simulate the effects of any of these therapeutic interventions in the context of AT lowering. In some embodiments, the QSP model can be used to determine an appropriate in-human dosage or dose regimen of a therapeutic intervention. The therapeutic intervention can rebalance hemostasis in people with HEMA and/or HEMB. The QSP model enables these determinations without requiring further human testing thereby providing information which may be impractical or impossible to clinically obtain. In some embodiments, the QSP model can be implemented with a virtual population to execute a virtual clinical trial to evaluate the effects of a therapeutic intervention. The present disclosure has determined that such techniques can facilitate development of new and more effective treatment modalities for rebalancing hemostasis in people with HEMA and/or HEMB. Accordingly, some aspects provide for a computer-implemented method for modeling thrombin-antithrombin interactions and thrombin-α-2-macroglobulin interactions after administration of fitusiran and/or supplemental clotting factor, comprising: obtaining a quantitative systems pharmacology (QSP) model representing, among other things, thrombin-antithrombin interactions and thrombin-α-2-macroglobulin interactions including a mechanism by which fitusiran targets and lowers antithrombin levels; determining thrombin level descriptors; assigning the thrombin level descriptors to a virtual patient population; and processing the virtual patient population using the QSP model to provide processed data, wherein the processed data comprises a concentration of at least one biomarker (e.g., results of a simulated thrombin generation assay and/or activated partial thromboplastin time). In some embodiments, the method further comprises displaying the processed data; determining therapeutic intervention data based on administration of one or more administered therapeutics; and processing the therapeutic intervention data and the virtual patient population with the QSP model to determine effectiveness of the administered one or more therapeutics. In some embodiments, the administration of the administered therapeutic comprises administration of fitusiran and/or supplemental clotting factor, e.g., Factor VIII.

In some embodiments, the disclosure provides methods for modeling hemostatic equivalency of fitusiran prophylaxis, particularly relating to thrombin generation in severe hemophilia A or severe hemophilia B in the context of antithrombin (AT) lowering. In some embodiments, the disclosure provides methods for modeling hemostatic equivalency of fitusiran prophylaxis, particularly relating to thrombin generation in moderate hemophilia A or moderate hemophilia B in the context of antithrombin (AT) lowering. In some embodiments, the disclosure provides methods for modeling hemostatic equivalency of fitusiran prophylaxis, particularly relating to thrombin generation in mild hemophilia A or mild hemophilia B in the context of antithrombin (AT) lowering.

In some embodiments, the QSP model represents binding/unbinding between thrombin and antithrombin. The QSP model can simulate a conversion of unbound thrombin and antithrombin to a thrombin-antithrombin complex.

In some embodiments, the QSP model comprises parameters including, but not limited to, plasma concentration of α-2-macroglobulin; degradation or removal rate of α-2-macroglobulin; synthesis rate of α-2-macroglobulin; binding rate of α-2-macroglobulin and thrombin; K_{D} of α-2-macroglobulin and thrombin; dissociation rate of α-2-macroglobulin and thrombin; catalysis of S2238 by thrombin; and catalysis of S2238 by the α-2-macroglobulin-thrombin complex.

In some embodiments, the method further comprises using the processed data to determine a change in the concentration of at least one biomarker over time. In some embodiments, the QSP model comprises a plurality of differential equations representing one or more biological reactions.

### Hemophilia

Hemophilia A (HEMA) is an X-linked recessive bleeding disorder caused by a deficiency in the activity of coagulation factor VIII. The disorder is clinically heterogeneous with variable severity, depending on the plasma levels of coagulation Factor VIII: mild, with levels about 6% to about 30% of normal Factor VIII levels; moderate, with Factor VIII levels about 2% to about 5% of normal; and severe, with Factor VIII levels less than about 1% of normal. Patients with mild hemophilia can bleed excessively only after trauma or surgery, whereas those with severe hemophilia can have an annual average of 20 to 30 episodes of spontaneous or excessive bleeding after minor trauma, particularly into joints and muscles. These symptoms differ substantially from those of bleeding disorders due to platelet defects or von Willebrand disease, in which mucosal bleeding predominates. Features of the disease and its etiology are reviewed in, for example, Mannucci P M, Tuddenham E G. The hemophilias—from royal genes to gene therapy. N Engl J Med. 2001 Jun. 7;344(23): 1773-9., which is incorporated herein by reference in its entirety.

The severity and frequency of bleeding in hemophilia A is inversely related to the amount of residual factor VIII in the plasma: less than 1% factor VIII results in severe bleeding, 2% to 6% results in moderate bleeding, and 6% to 30% results in mild bleeding. The proportion of cases that are severe, moderate, and mild are about 50%, 10%, and 40%, respectively. The joints can be affected, causing swelling, pain, decreased function, and degenerative arthritis. Similarly, muscle hemorrhage can cause necrosis, contractures, and neuropathy by entrapment. Hematuria can occur and can be painless. Intracranial hemorrhage can occur after even mild head trauma and can lead to severe complications. Bleeding from tongue or lip lacerations can be persistent.

Hemophilia B due to factor IX deficiency is phenotypically comparable to hemophilia A, which as described above results from deficiency of coagulation factor VIII. The classic laboratory findings in hemophilia B include a prolonged activated partial thromboplastin time (aPTT) and a normal prothrombin time (PT).

### Fitusiran

The structure of fitusiran is provided herein. Fitusiran is a synthetically, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. See, e.g., Pasi, supra. Antithrombin is encoded by the SERPINC1 gene. The nucleosides in each strand of fitusiran are connected through either 3′-5′ phosphodiester or phosphorothioate linkages, thus forming the sugar-phosphate backbone of the oligonucleotide.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH).

The sense strand and the antisense strand of fitusiran contain 21 and 23 nucleotides, respectively. The 3′ end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, which describe fitusiran and its use in methods of treating hemophilia. The entire contents of each of these references are expressly incorporated herein by reference.

The two nucleotide strands of fitusiran are shown below:
sense strand: 5′Gf-ps-Gm-ps-Uf-Um-Af-Am-Cf-Am-Cf-Cf-Af-Um-Uf-Um-Af-Cm-Uf-Um-Cf-Am-Af-L96 3′ (SEQ ID NO:1), and
antisense strand: 5′ Um-ps-Uf-ps-Gm-Af-Am-Gf-Um-Af-Am-Af-Um-Gm-Gm-Uf-Gm-Uf-Um-Af-Am-Cf-Cm-ps-Am-ps-Gm 3′ (SEQ ID NO:2),
wherein
   Af=2′-fluoroadenosine (i.e., 2′-deoxy-2′-fluoroadenosine)
   Cf=2′-fluorocytidine (i.e., 2′-deoxy-2′-fluorocytidine)
   Gf=2′-fluoroguanosine (i.e., 2′-deoxy-2′-fluoroguanosine)
   Uf=2′-fluorouridine (i.e., 2′-deoxy-2′-fluorouridine)
   Am=2′-O-methyladenosine
   Cm=2′-O-methylcytidine
   Gm=2′-O-methylguanosine
   Um=2′-O-methyluridine
   “-” (hyphen)=3′-5′ phosphodiester linkage sodium salt
   “-ps-”=3′-5′ phosphorothioate linkage sodium salt and wherein L96 has the following formula:

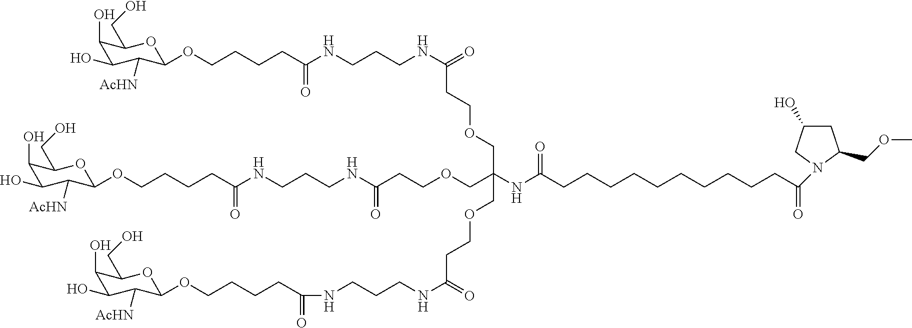 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

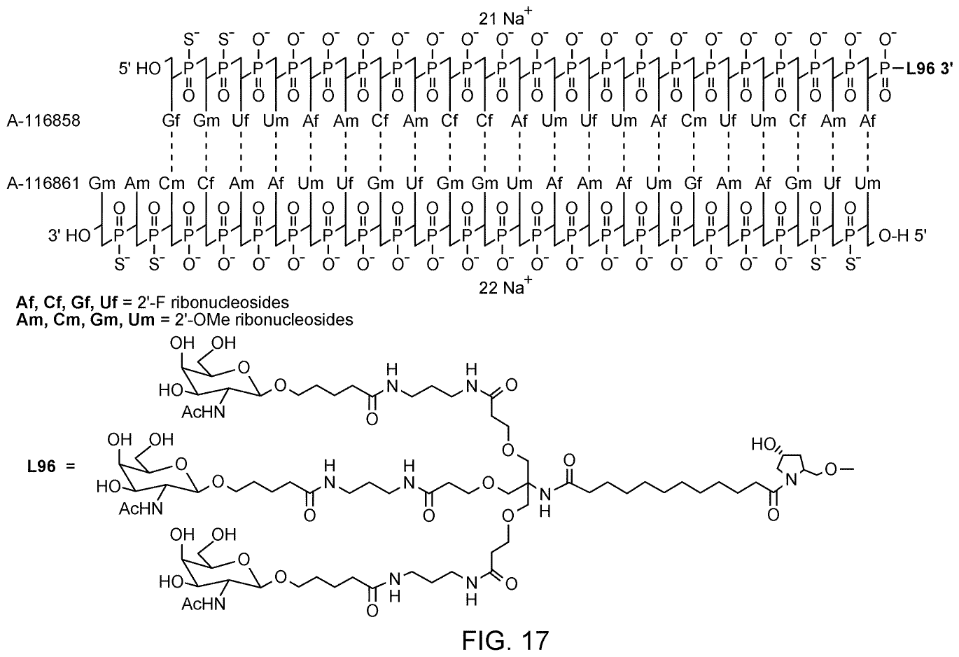

The expanded structural formula, molecular formula, and molecular weight of fitusiran are shown in FIG. 17.

The structure of fitusiran can also be described using the following diagram, wherein the X is O:

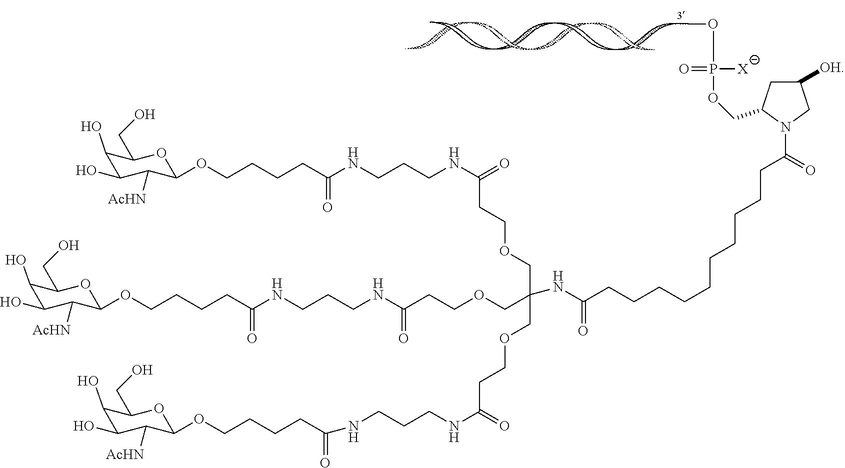

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3):386-9). Fitusiran may be used to treat those who have impaired hemostasis. For example, fitusiran can be used to treat patients with hemophilia A or B, with or without inhibitors for routine prophylaxis to prevent or reduce the frequency of bleeding episodes. In particular embodiments, fitusiran is used to treat patients, for example adult and adolescent patients (≥12 years of age), with hemophilia A or B (congenital factor VIII or factor IX deficiency), with or without inhibitors.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A hemophilia A or B patient without inhibitors refers to a patient who does not have such alloantibodies.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

The present methods include administering to the hemophilia patient (e.g., a hemophilia A or B patient, with or without inhibitors) in need thereof a prophylactically effective amount of fitusiran. “Prophylactically effective amount” refers to the amount of fitusiran that helps the patient with hemophilia A or B, with or without inhibitors, to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint. The term “prophylaxis” and “prophylactic treatment” are used interchangeably herein.

In some embodiments, a prophylactically effective amount of fitusiran is about 20 to about 80 mg of fitusiran (e.g., about 20 mg, about 25 mg, about 30 mg, about 40 mg, about 50 mg, or about 80 mg). In some embodiments, a prophylactically effective amount of fitusiran is about 1 to about 30 mg of fitusiran (e.g., about 1.25 mg, about 2.5 mg, about 5 mg, about 10 mg, about 20 mg, or about 30 mg).

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

The prophylactically effective amount of fitusiran may be delivered about every month (or about every four weeks) or about once every two months (or about every eight weeks).

AT measurements can be performed by well-established methods, including both kinetic and chromogenic assays. The AT activity (%) in a plasma sample is calculated against the WHO reference plasma. 100% AT level is defined as 1 unit of antithrombin activity in 1 mL of reference plasma sample. AT levels range from about 80% to about 120% in the general population.

It has been observed that the risk of arterial thrombotic events among patients receiving fitusiran may increase with AT levels <10%. To mitigate the risk of vascular thrombotic events while maintaining a favorable benefit-risk balance for patients on fitusiran, a patient, for example an adult patient (≥18 years of age) or an adolescent patient (12 to 17 years of age, inclusive), may start on a fitusiran therapy by subcutaneous injection of 50 mg fitusiran every two months (or every eight weeks). The patient's AT level is monitored periodically (e.g., every one, two, three, four, five, six, seven, or eight weeks, or every one, two, three, four, five, or six months). If the patient has two AT measures of <15% (e.g., <10%), the patient will discontinue fitusiran treatment. In some embodiments, upon the first AT level <15%, the patient has another AT activity level sample drawn within a month (e.g., within one or two weeks). If this result is <15%, this will be considered the second AT <15%. Patients receiving fitusiran at a dose of 50 mg Q2M with more than 1 (e.g., 2) AT activity levels <15% will discontinue fitusiran.

However, if the 50 mg/Q2M (or Q8W) patient has two AT measures of >25% (e.g., >35%), the patient will escalate the dosing regimen. The patient may receive fitusiran at 50 mg every month (or every four weeks); if the patient has two AT measurements of >25% (e.g., >35%) under the 50 mg/QM (Q4W) regimen, the patient may receive fitusiran at 80 mg every month (or every four weeks). In some cases, the 50 mg/Q2M (or Q8W) patient may receive fitusiran at 80 mg every two months (or every eight weeks); if the patient has two AT measurements of >25% (e.g., 35%) under the 80 mg/Q2M (or Q8W) regimen, the patient may receive fitusiran at 50 mg every month (or every four weeks); if he has two AT measurements of >25% (e.g., 35%) under the 50 mg/QM (or Q4W) regimen, he will receive fitusiran at 80 mg every month (or every four weeks). In some cases, the 50 mg/Q2M (or Q8W) patient may receive fitusiran at 80 mg every two months (or every eight weeks); if the patient has two AT measurements of >25% (e.g., >35%) under the 80 mg/Q2M (or Q8W) regimen, the patient may receive fitusiran at 80 mg every month (or every four weeks).

Patients who have discontinued fitusiran after having more than one (e.g., 2) AT activity levels <15% when receiving fitusiran at a dose of 50 mg Q2M may receive fitusiran at a dose of 20 mg Q2M once their AT levels have returned to ≥22%. Patients receiving fitusiran at a dose of 20 mg Q2M with more than 1 (e.g., 2) AT activity level <15% will discontinue fitusiran treatment. If a patient receiving fitusiran at a dose of 20 mg/Q2M (or Q8W) has two AT measures of >25% (e.g., >35%), the patient may receive fitusiran at 20 mg QM or Q4W.

In the above dose finding regimens, AT measurements for dosing determination are those taken during steady state (SS) of AT activity, i.e., once the patient's AT levels have been stabilized (at low AT activity range) after fitusiran treatment. The SS is typically reached after two or three doses of fitusiran. AT measurements for dosing determination are taken at an appropriate interval (e.g., every four weeks or every eight weeks).

In the above dose finding regimens, the starting dose of 50 mg fitusiran Q2M is included as an illustrative example. For example, a starting dose of fitusiran may be 50 mg Q2M, 20 mg Q2M, 20 mg QM, or 10 mg QM. Dose escalation and de-escalation can then be carried out accordingly from each starting dose. For example, a starting dose of 20 mg Q2M fitusiran can be escalated to 20 mg QM, 50 mg Q2M, 50 mg QM, or 80 mg QM, optionally sequentially in that order, or de-escalated to 10 mg QM.

An AT level of 10-35% (e.g., 10-25%, 15-35%, or 15-25%) is targeted to mitigate the risk of vascular thrombotic events while maintaining a favorable benefit-risk balance for patients on fitusiran. Thus, so long as the patient reaches this targeted AT level, there is no need for the patient to receive a higher fitusiran dosage or more frequent dosing. That is, he remains on the current treatment regimen (i.e., maintenance regimen). For example, once the desired AT level is reached, the patient may be treated with a subcutaneous dose of fitusiran (e.g., 40-90 mg per dose) at an interval of, e.g., every one, two, three, four, five, six, seven, or eight weeks, or every one, two, three, four, five, or six months. In some embodiments, if the patient has two AT measurements of no greater than 35% while receiving 50 mg Q2M, he will maintain this dosing regimen, with no need to further escalate the dosage or dosing frequency. As another example, if the patient has two AT measurements of no greater than 35% while receiving 80 mg Q2M or 50 mg QM, he will remain on this dosing regimen, with no need to further escalate the dosage or dosing frequency (to, e.g., 80 mg QM). However, fitusiran treatment should be discontinued if a patient has more than 1 (e.g., 2) AT measurements <15% (e.g., <10%) as a risk mitigation measure for vascular thrombotic events. Alternatively, the patient may resume treatment with a lower dose of fitusiran after their AT levels have returned to above 15%, e.g., ≥22%.

A detailed description of fitusiran and its use thereof can be found e.g., in WO2022/120292, which is incorporated herein by the reference in its entirety.

### QSP Modeling

QSP modeling is a mechanistic approach that integrates clinical and nonclinical data of, in the context of the present disclosure, the coagulation pathway, to predict the results of multiple in vitro coagulation assays, including the thrombin generation assay (TGA), and to mechanistically understand the readouts of these assays and of hemostatic equivalency of fitusiran prophylaxis (i.e., AT lowering). The QSP model disclosed herein also models the coagulation cascade and represents reported steady-state levels of coagulation factors and accessory proteins in the plasma of healthy individuals and people with hemophilia A (PwHA) and people with hemophilia B (PwHB). In some embodiments, the model also includes parameters describing the impact of α-2-macroglobulin, a key regulator of thrombin, with increased influence at reduced AT levels to provide predictions relating to the impact of fitusiran on thrombin generation.

To this end, the QSP models disclosed herein account for properties specific to fitusiran, antithrombin, thrombin, antithrombin lowering, the thrombin-antithrombin complex, α-2-macroglobulin, the α-2-macroglobulin-thrombin complex, fibrinogen, and fibrin and fibrin degradation products. Example of such properties include, but are not limited to, plasma concentration of α-2-macroglobulin, degradation or removal rate of α-2-macroglobulin, synthesis rate of α-2-macroglobulin, binding rate of α-2-macroglobulin and thrombin, K_{D} of α-2-macroglobulin and thrombin, dissociation rate of α-2-macroglobulin and thrombin, catalysis of S238 by thrombin, and catalysis of S2238 by the α-2-macroglobulin-thrombin complex.

The QSP models disclosed herein consider, among other things, peak thrombin levels, predicted peak height in a thrombin generation assay, area under the curve (AUC) in a thrombin generation assay, and lag time in a thrombin generation assay in samples from subjects administered fitusiran and/or supplemental Factor VIII, in plasma samples from subjects administered fitusiran and/or supplemental Factor VIII.

In some embodiments, the QSP models disclosed herein predict peak thrombin levels, predicted peak height in a thrombin generation assay, area under the curve (AUC) in a thrombin generation assay, and lag time after subjects are administered fitusiran and/or supplemental Factor VIII by considering some or all of the following factors as inputs: AT synthesis in plasma, synthesis of coagulation factors and coagulation proteins in plasma, elimination of coagulation factors and coagulation proteins in plasma. In some embodiments, a simulated plasma sample of a subject considers some or all of the following factors: AT concentration, factor II concentration, factor V concentration, factor VII concentration, factor VIII concentration, factor IX concentration, factor X concentration, factor XI concentration, factor XII concentration, α-2-macroglobulin concentration, Protein S concentration, Protein C concentration, thrombomodulin concentration, and prekallikrein concentration.

### Flow Charts

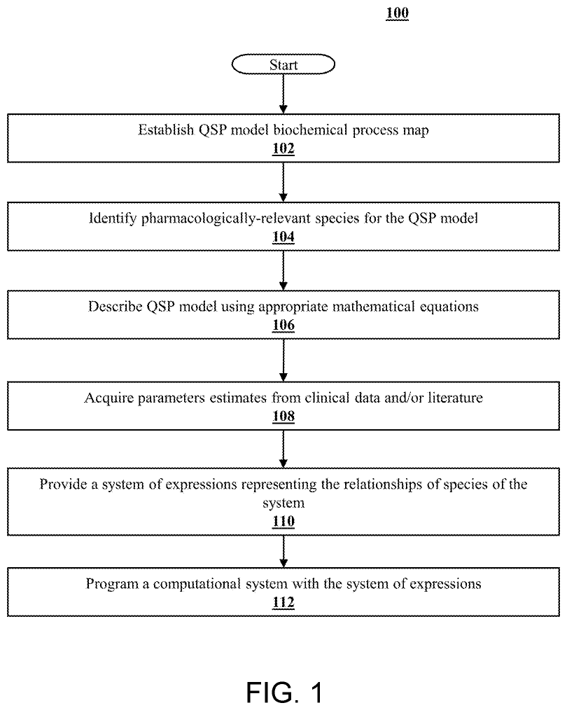

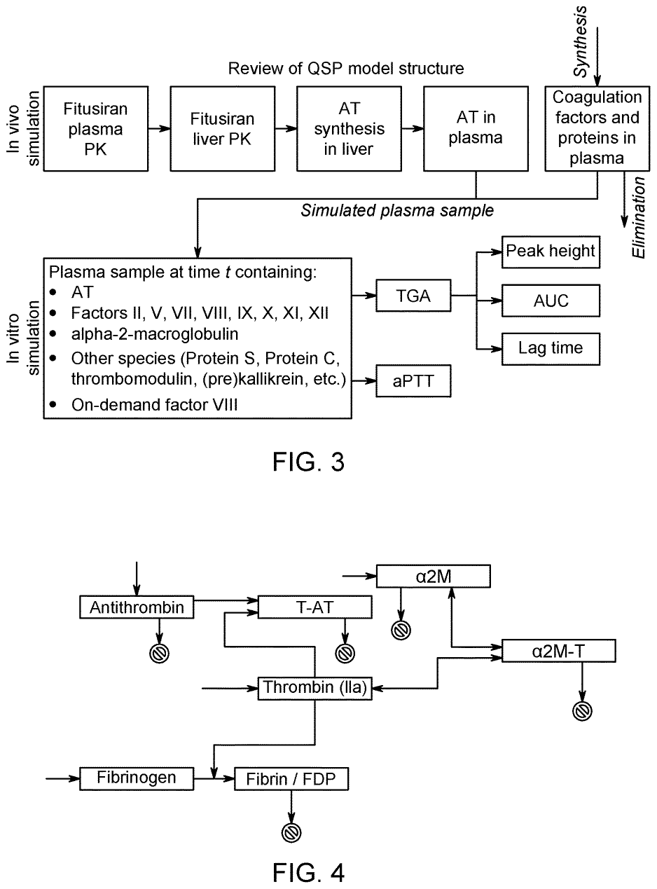

FIG. 1 presents a flow chart for an example method of generating a QSP model for predicting peak thrombin levels, predicted peak height in a thrombin generation assay, area under the curve (AUC) in a thrombin generation assay, and lag time after subjects are administered fitusiran and/or supplemental Factor VIII. The method is represented by reference numeral 100 and begins with an operation 102 in which a biochemical process map is generated. The biochemical process maps for the QSP model disclosed herein are depicted in, for example, FIG. 3 and FIG. 4.

In operation 104, pharmacologically-relevant species for the QSP model are identified. Pharmacologically-relevant species for the QSP model disclosed herein are provided in, for example, Table 1 below.

TABLE 1
List of species in the QSP model

| Species Name | Units | Description |
|---|---|---|
| Antithrombin | nM | Amount of antithrombin in plasma |
| Thrombin- | nM | Amount of thrombin complexed with |
| Antithrombin | | antithrombin in plasma |
| α-2-macroglobulin | nM | Amount of α-2-macroglobulin in plasma |
| Thrombin-α-2- | nM | Amount of thrombin complexed with α-2- |
| macroglobulin | | macroglobulin in plasma |
| Thrombin (IIa) | nM | Amount of thrombin in plasma |
| Fibrinogen | nM | Amount of fibrinogen in plasma |
| Fibrin and/or FDP | nM | Amount of fibrin and/or fibrin degradation |
| | | products in plasma |
| Factor II | nM | Amount of Factor II in plasma |
| Factor V | nM | Amount of Factor V in plasma |
| Factor VII | nM | Amount of Factor VII in plasma |
| Factor VIII | nM | Amount of Factor VIII in plasma |
| Factor IX | nM | Amount of Factor IX in plasma |
| Factor X | nM | Amount of Factor X in plasma |
| Factor XI | nM | Amount of Factor XI in plasma |
| Factor XII | nM | Amount of Factor XII in plasma |
| Protein S | nM | Amount of Protein S in plasma |
| Protein C | nM | Amount of Protein C in plasma |
| Thrombomodulin | nM | Amount of thrombomodulin in plasma |
| Prekallikrein | nM | Amount of prekallikrein in plasma |

With the in vivo simulation, simulated plasma sample, and in vitro simulations and relevant species identified, the computer system receives a set of relationships representing pharmacokinetics, pharmacodynamics and/or reactions of the species in the subject and in the simulated plasma sample. Exemplary reactions between species for the QSP model disclosed herein are provided in, for example, Table 2 below.

TABLE 2
List of reactions in the model
Reactions

| | |
|---|---|
| Binding/unbinding between | T + AT ↔ T − AT |
| thrombin and antithrombin | |
| Binding/unbinding between | α2M + T ↔ α2M − T |
| α-2-macroglobulin and | |
| thrombin | |
| Conversion of fibrinogen | Fibrinogen + T → fibrin + FDP |
| to fibrin and fibrin | |
| degradation products by | |
| thrombin | |
| r_prod_IIa ATIII | k_prod_IIa_ATIII * x(index_IIa) * |
| | x(index_ATIII); |
| r_bind_Fg_IIa | k_on_IIa_Fg * x(index_Fg) * |
| | x(index_IIa) − k_off_IIa_Fg * |
| | x(index_IIa_Fg); |
| r_prod_F_by_IIa | kcat_Fg_to_F * x(index_IIa_Fg); |
| r_bind_XIIIa_F | k_on_XIIIa_F * x(index_XIIIa) * |
| | x(index_F) − x_off_XIIIa_F * |
| | x(index_XIII_F); |
| r_bind_F_P | k_on_P_F * x(index_F) * x(index_P) − |
| | k_off_P_F * x(index_P_F); |
| r_syn_Fg | ksyn_Fg; |
| r_syn_deg_ATIII | (k_syn_AT_mod * mod_AT_synthesis) − |
| | k_deg_AT_mod * x(index_ATIII); |
| r_deg_IIa_ATIII | kdeg_IIa_ATIII * x(index_IIa_ATIII); |
| r_deg_FDP | kdeg_FDP * x(index_FDP); |
| r_syn_deg_a2M | ksyn_a2M − kdeg_a2M * |
| | x(index_a2M); |
| r_bind_a2M_IIa | k_bind_a2M_II * x(index_a2M) * |
| | x(index_IIa) − k_off_a2M_II * |
| | x(index_a2M_IIa); |
| r_deg_a2M_IIa | (1 − in_vitro_tag) * kdeg_a2M * |
| | x(index_a2M_IIa); |

These reactions and relationships between species can be described by one or more equations, and the QSP model is described in operation 106 using appropriate governing mathematical equations. Equations for the QSP model disclosed herein are provided in, for example, Table 3 below.

TABLE 3
List of governing equations in the model

| | |
|---|---|
| dxdt(index_IIa) | 1/blood * (r_act_IIa_by_Xa + |
| | r_cat_Xa_Va_II + |
| | r_prod_IIa_from_mIIa + −r_{—} |
| | prod_IIa_ATIII + −r_prod_IIa_{—} |
| | Tmod + −r_bind_Fg_IIa + r_prod_F_by_{—} |
| | IIa + −r_bind_XIII_IIa + r_prod_{—} |
| | XIIIa + −r_bind_a2M_IIa); |
| dxdt(index_ATIII) | 1/blood * (r_syn_deg_ATIII + −r_{—} |
| | prod_Xa_ATIII + −r_prod_mIIa_{—} |
| | ATIII + −r_{—} |
| | prod_IXa_ATIII + −r_{—} |
| | prod_IIa_ATIII + −r_{—} |
| | prod_TF_VIIa_ATIII + −r_{—} |
| | prod_XIIa_ATIII + −r_{—} |
| | prod_XIa_ATIII + −r_{—} |
| | prod_VIIa_ATIII + −r_{—} |
| | prod_VIIa_ATIII_supp); |
| dxdt(index_IIa_ATIII) | 1/blood * (−r_deg_IIa_ATIII + |
| | r_prod_IIa_ATIII); |
| dxdt(index_Fg) | 1/blood * (r_syn_Fg + −r_bind_Fg_{—} |
| | IIa + −r_bind_Fg_P); |
| dxdt(index IIa_Fg) | 1/blood * (r_bind_Fg_IIa + −r_{—} |
| | prod_F_by_IIa); |
| dxdt(index_F) | 1/blood * (r_prod_F_by_IIa + −r_{—} |
| | bind_XIIIa_F + −r_bind_F_P); |
| dxdt(index_FDP) | 1/blood * (−r_deg_FDP + |
| | r_deg_Fg_by_P + r_deg_F_by_P); |
| dxdt(index_a2M) | 1/blood * (r_syn_deg_a2M + −r_{—} |
| | bind_a2M_IIa); |
| dxdt(index_a2M_IIa) | 1/blood * (r_bind_a2M_IIa + −r_{—} |
| | deg_a2M_IIa); |

Subsequent to developing the model diagram of the QSP model and set of differential equations representing reactions between parameters reflected in the model, the QSP model can be parameterized in operation 108. For example, parameters of the model, defined in Table 4 below, can be set to initial values based on either literature data, clinical data, known mathematical relationships between other parameters, or obtained via further calibration steps described herein. Table 4 below gives the model parameters, their initial values, and the source for the initial values.

TABLE 4
List of model parameters

| Parameters | Values | Units | Description |
|---|---|---|---|
| α2M | [3, 6] | μM | Plasma concentration of α-2- |
| | | | macroglobulin |
| kdeg_{α2M} | [8.4, 21] | hr⁻¹ | Degradation or removal rate of |
| | | | α-2-macroglobulin |
| ksyn_{α2M} | [25.2, 126] | μM/hr | Synthesis rate of α-2- |
| | | | macroglobulin |
| kon_{α2M-IIa} | [5, 8.7]⁻⁷ | nM⁻¹s⁻¹ | Binding rate of α-2- |
| | | | macroglobulin and thrombin |
| kD_{α2M-IIa} | 7.9⁻⁸ | M | K_{D} of α-2-macroglobulin and |
| | | | thrombin |
| koff_{α2M-IIa} | [0.4, 0.7]⁻⁴ | s⁻¹ | Dissociation rate of α-2- |
| | | | macroglobulin and thrombin |
| kcat_{IIa} | 1.9 | M⁻¹s⁻¹ | Catalysis of S238 by thrombin |
| kcat_{α2M-IIa} | 0.9 | M⁻¹s⁻¹ | Catalysis of S238 by the α-2- |
| | | | macroglobulin-thrombin |
| | | | complex |

These relationships can include rate constants, equilibrium constants, concentrations of one or more species, etc. In certain embodiments, one or more of these relationships provide the rate of accumulation or depletion of a species due to a particular physical phenomenon (e.g., synthesis, degradation or a reaction within a compartment). In some embodiments, one or more of the relationships is a ratio of concentrations of two or more species or a ratio of products of these species (e.g., an equilibrium constant or partition coefficient). In certain embodiments, the computer system obtains parameters such as rate constants for these relationships. Examples of sources of these parameters and methods of determining them are provided below.

With the relationships received, the computer system uses the rate constants, species concentrations, and any other components of the relationships to produce a system of expressions that can be used by the computational system to execute the QSP model. See operation 110. In certain embodiments, this operation includes organizing information from the set of relationships into, vectors, matrices, tensors, specified data structures, and/or other constructs that the computer system can use to calculate a time-dependent concentration of one or more species over a defined duration. The system of expressions is generally a computer-useable representation of the equations or other mathematics characterizing species in compartments. In certain embodiments, the system of expressions includes expressions representing one or more differential equations for the in vivo simulation, simulated plasma sample, and in vitro simulations.

With the system of expressions provided, the computer system programs a particular computational system with the system of expressions in a form ready for execution. See operation 112. In some cases, the computer system used to generate the QSP model is the same as the particular computational system programmed to execute the model. In other cases, the two systems are different, physically or logically. The programming of operation 112 allows the computational system to execute the QSP model when provided with appropriate initial conditions (pharmacological conditions) or other information.

Receiving instructions or data in operations 106, 108, 110 and/or 112 refers to actions of by or for a computer system that generates the QSP model. These actions can include inputting and/or storing information in memory accessible by processors responsible for programming computational system with instructions and data that comprise the QSP model. A human user can be indirectly responsible for causing a transmission of instructions and/or data to the portion of the computational system where it can be used to program the QSP model.

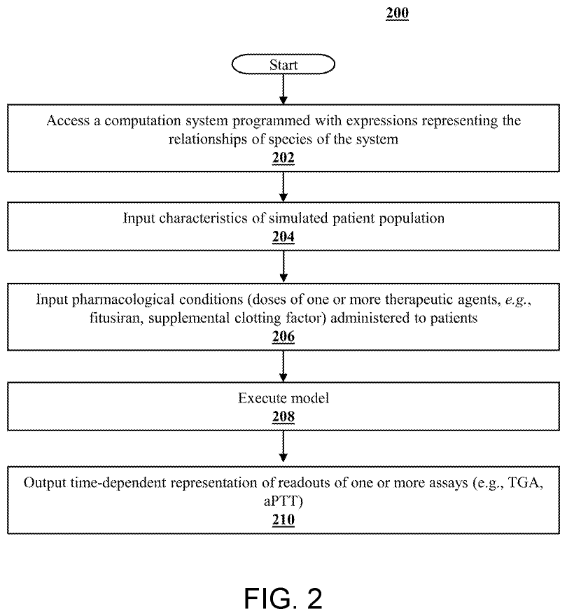

FIG. 2 presents a flow chart for an example method of using a QSP model to predicting peak thrombin levels, predicted peak height in a thrombin generation assay, area under the curve (AUC) in a thrombin generation assay, and lag time after subjects are administered fitusiran and/or supplemental Factor VIII. The method is represented by reference numeral 200 and begins with an operation 202 in which a computational system used in executing the QSP model is accessed or otherwise made available for execution. In certain embodiments the QSP model is generated using a method following the process of FIG. 1 followed by execution as depicted in FIG. 2. Regardless, the computational system is programmed with expressions representing concentration and/or reaction parameters involving, for example, fitusiran, Factor VIII, and antithrombin in a simulated plasma sample of a subject.

With the QSP model available, the computational system can receive and/or input various data and/or commands necessary to execute the model in a way that predicts peak thrombin levels, predicted peak height in a thrombin generation assay, area under the curve (AUC) in a thrombin generation assay, and lag time after subjects are administered fitusiran and/or supplemental Factor VIII. and/or PK/PD of fitusiran and/or supplemental Factor VIII. For example, the computational system can receive and/or input properties specific for a particular simulated patient population. See operation 204. Examples of such properties include biochemical characteristics of enzymes and substrates including thrombin, antithrombin, α-2-macroglobulin, and fibrinogen; e.g., synthesis and/or degradation rates of the enzymes and substrates, binding rates of the enzymes and substrates, and dissociation rates of the enzymes and substrate. This information can be provided various forms such as binding rates, cleavage rates, catalysis rates, K_{D}, and the like for simulated patients or for a simulated patient population.

In an operation 206, the computational system receives or inputs conditions of a subject who is to be administered fitusiran and/or supplemental Factor VIII. In some embodiments, the subject is known to have hemophilia A or hemophilia B. In some embodiments, these inputs include information such as the mass of the subject characteristics and the disease state of the subject. The intrinsic parameters can be prescribed for each simulated patient or for the simulated patient population.

The computational system further receives or inputs one or more pharmacological conditions associated with administering the fitusiran and/or supplemental Factor VIII to the subject. Such pharmacological conditions are sometimes referred to as extrinsic parameters. As explained herein, such parameters concern the subject's treatment and they can include various details about how the fitusiran and/or supplemental Factor VIII is administered to the subject; e.g., doses in a treatment regimen.

With the intrinsic and extrinsic parameters available, the QSP model is ready to execute. Execution is depicted in operation 208 and involves performing various mathematical or numerical operations on the data and/or commands received via operations 106, 108, 110, 112, 202, 204, and 206. The mathematical or numerical operations are performed by following instructions for, e.g., solving a system of expressions such as generated in operation 110.

During or after execution, the computational system outputs values relevant to the peak thrombin levels, predicted peak height in a thrombin generation assay, area under the curve (AUC) in a thrombin generation assay, and lag time after subjects are administered fitusiran and/or supplemental Factor VIII. See operation 210. These values can be time-dependent representations of thrombin levels in plasma of the subject, or can be values that influence thrombin levels or downstream coagulation parameters in plasma of the subject. In certain embodiments, the values are PD or PK parameters of fitusiran and/or supplemental Factor VIII. In certain embodiments, the values are target therapeutic ranges of fitusiran and/or supplemental Factor VIII doses.

### Components of the QSP Model

### Thrombin

Thrombin is a serine protease, an enzyme that, in humans, is encoded by the F2 gene. The thrombin precursor prothrombin (coagulation factor II) is proteolytically cleaved to form thrombin in the clotting process. Thrombin in turn acts as a serine protease that converts soluble fibrinogen into insoluble strands of fibrin which promotes the clotting process. Thrombin also catalyzes many other coagulation-related reactions, including converting Factor XI to XIa, Factor VIII to VIIIa, Factor V to Va, and Factor XIII to XIIIa, and stimulates platelet aggregation. The molecular weight of prothrombin is approximately 72 kDa. The catalytic domain is released from prothrombin fragment 1.2 to create the active enzyme thrombin, which has a molecular weight of 36 kDa. Prothrombin is composed of four domains; an N-terminal Gla domain, two kringle domains and a C-terminal trypsin-like serine protease domain. Prothrombin is converted to active thrombin by proteolysis of an internal peptide bond, exposing a new N-terminal Ile-NH3.

### Antithrombin

Antithrombin (AT), a small glycoprotein, is a plasma protease inhibitor and a member of the serpin superfamily. This protein inhibits thrombin as well as other activated serine proteases of the coagulation system, and it regulates the blood coagulation cascade. The antithrombin protein includes two functional domains: the heparin binding-domain at the N-terminus of the mature protein, and the reactive site domain at the C-terminus. AT is a 432-amino-acid protein produced by the liver. It contains three disulfide bonds and a total of four possible glycosylation sites. α-antithrombin is the dominant form of antithrombin found in blood plasma and has an oligosaccharide occupying each of its four glycosylation sites. A single glycosylation site remains consistently un-occupied in the minor form of antithrombin, β-antithrombin.

The physiological target proteases of antithrombin are those of the contact activation pathway (formerly known as the intrinsic pathway), namely the activated forms of Factor X (Xa), Factor IX (IXa), Factor XI (XIa), Factor XII (XIIa) and, to a greater extent, Factor II (thrombin) (IIa), and also the activated form of Factor VII (VIIa) from the tissue factor pathway (formerly known as the extrinsic pathway). AT also inactivates kallikrein and plasmin, also involved in blood coagulation. However it inactivates certain other serine proteases that are not involved in coagulation such as trypsin and the C1s subunit of the enzyme C1 involved in the classical complement pathway.

Protease inactivation by AT results as a consequence of trapping the protease in an equimolar complex with antithrombin in which the active site of the protease enzyme, for example thrombin, is inaccessible to its usual substrate. The formation of an antithrombin-protease complex, for example an antithrombin-thrombin complex, involves an interaction between the protease and a specific reactive peptide bond within antithrombin. For human antithrombin this bond is between arginine (arg) 393 and serine (ser) 394.

Protease enzymes interacting with antithrombin can become trapped in inactive antithrombin-protease complexes as a consequence of their attack on the reactive bond. While not wishing to be bound by any particular theory, although attacking a similar bond within the normal protease substrate results in rapid proteolytic cleavage of the substrate, initiating an attack on the antithrombin reactive bond causes antithrombin to become activated and trap the enzyme at an intermediate stage of the proteolytic process. Over time time after association with antithrombin, thrombin is able to cleave the reactive bond within antithrombin and an inactive antithrombin-thrombin complex will dissociate, however the time it takes for this to occur may be greater than 3 days.

### Supplemental Factor VIII

Factor VIII is a blood-clotting protein, also known as anti-hemophilic factor (AHF). In humans, factor VIII is encoded by the F8 gene. Defects in this gene result in hemophilia A, a recessive X-linked coagulation disorder. Factor VIII is produced in liver sinusoidal cells and endothelial cells outside the liver throughout the body. This protein circulates in the bloodstream in an inactive form, bound to another molecule called von Willebrand factor until, for example, an injury that damages blood vessels occurs. In response to injury, coagulation factor VIII is activated and separates from von Willebrand factor. The active protein interacts with another coagulation factor called factor IX. This interaction can trigger a chain of additional chemical reactions that form a blood clot.

Supplemental Factor VIII can be used as a medication for patients with HEMA for the prevention and control of bleeding episodes after injury. Supplemental Factor VIII can also be used as a medication for the maintenance of hemostasis in patients with HEMA undergoing surgery (i.e., perioperative management). Factor VIII replacement therapy generally is required in patients with mild to moderate hemophilia A who do not respond adequately to desmopressin or those with moderate to severe HEMA and factor VIII levels <5% of normal. Supplemental Factor CIII is effective in the management of spontaneous or traumatic bleeding episodes (e.g., hemarthrosis, IM hematoma, soft tissue bleeding) or acute bleeding events (e.g., GI, retroperitoneal, tonsillar, ocular) in patients with HEMA. Supplemental Factor VIII can also used for routine prophylaxis (i.e., administration at regular intervals) to prevent or reduce frequency of bleeding events. Such prophylaxis is considered the current standard of care for patients with HEMA. Factor VIII prophylaxis decreases frequency of spontaneous musculoskeletal bleeding, preserves joint function, and improves quality of life for patients with HEMA.

α-2-macroglobulin

α-2-macroglobulin is a regulator of thrombin and can affect the impact of fitusiran on thrombin generation. α-2-macroglobulin is a 720 kDa plasma protein found in the blood. It is mainly produced by the liver, and also locally synthesized by macrophages, fibroblasts, and adrenocortical cells. In humans it is encoded by the A2M gene. α-2-macroglobulin acts as an antiprotease and is able to inactivate a variety of proteinases. It functions as an inhibitor of fibrinolysis by inhibiting plasmin and kallikrein. It functions as an inhibitor of coagulation by inhibiting thrombin. α2-macroglobulin can act as a carrier protein because it also binds to numerous growth factors and cytokines, such as platelet-derived growth factor, basic fibroblast growth factor, TGF-β, insulin, and IL-1β.

α-2-macroglobulin inhibits by steric hindrance. The mechanism involves protease cleavage of the bait region, a segment of α-2-macroglobulin that is particularly susceptible to proteolytic cleavage, which initiates a conformational change such that the αM collapses about the protease. In the resulting α-2-macroglobulin-protease complex, the active site of the protease is sterically shielded, thus substantially decreasing access to protein substrates. α2-Macroglobulin is able to inactivate a variety of proteinases (including serine-, cysteine-, aspartic- and metalloproteinases), e.g., thrombin.

### Thrombin Generation Assay

A thrombin generation assay (TGA) or thrombin generation test (TGT) is a global coagulation assay (GCA) and type of coagulation test which can be used to assess coagulation and thrombotic risk. It is based on the potential of a plasma to generate thrombin over time, following activation of coagulation via addition of phospholipids, tissue factor, and calcium. The results of the TGA can be output as a thrombogram or thrombin generation curve using computer software with calculation of thrombogram parameters. TGAs can be performed with methods like, for example, the semi-automated calibrated automated thrombogram (CAT) or a fully-automated, for example, the ST Genesia system. TGAs were first used as manual assays in the 1950s and have since become increasingly automated.

In some embodiments, the QSP model disclosed herein performs a simulated TGA on a simulated plasma sample. In some embodiments, the simulated TGA of the QSP model receives as an input, parameters and values including, but not limited to, AT concentration, factor II concentration, factor V concentration, factor VII concentration, factor VIII concentration, factor IX concentration, factor X concentration, factor XI concentration, factor XII concentration, α-2-macroglobulin concentration, Protein S concentration, Protein C concentration, thrombomodulin concentration, and prekallikrein concentration. In some embodiments, the simulated TGA of the QSP model is performed for a simulated sample of a healthy individual. In some embodiments, the simulated TGA of the QSP model is performed for a simulated sample of an individual having hemophilia A. In some embodiments, the simulated TGA of the QSP model is performed for a simulated sample of an individual having hemophilia B. In some embodiments, the simulated TGA of the QSP model is performed for a simulated sample of an individual having hemophilia A or B after treatment with fitusiran. In some embodiments, the simulated TGA of the QSP model is performed for a simulated sample of an individual having hemophilia A or B after treatment with fitusiran and supplemental Factor VIII.

### Reaction Details

The QSP model disclosed herein uses various relationships and other details for relating to species of the model and reactions affecting the concentration of species of the model in, for example, simulated in vivo conditions of a subject, a simulated plasma sample of a subject, and simulated in vitro assays of a plasma sample of a subject. For example, the QSP model can be based on, among other things, mechanisms of association or dissociation of the thrombin-antithrombin complex, synthesis rate of α-2-macroglobulin, binding rate of α-2-macroglobulin and thrombin, or cleavage of fibrinogen by thrombin. In certain embodiments, reactions are modeled with 0th, 1st, and 2nd order mass action relationships.

Examples of relationships that can be used in QSP models follow. In certain embodiments, a QSP model employs any one or more of these relationships. In certain embodiments, a QSP model employs any two or more of these relationships.

### Expressions for Representation QSP Model in a Computational System

As discussed, the QSP model of the present disclosure executes instructions representing mathematical expressions characterizing one or more of the species in, for example, simulated in vivo conditions of a subject, a simulated plasma sample of a subject, and simulated in vitro assays of a plasma sample of a subject. The mathematical representation is provided as a set of expressions of the relationships and quantities for the species in the simulated in vivo conditions of the subject, the simulated plasma sample of the subject, and the simulated in vitro assays of the plasma sample of the subject. In some implementations, the simulated in vivo conditions of the subject, the simulated plasma sample of the subject, and the simulated in vitro assays of the plasma sample of the subject each have one or more separate mathematical expressions, with one for each species under consideration in the condition being modeled. The expressions correspond to the governing relationships, such as the reaction phenomena described herein. The mathematical representation can include all information sufficient for representing or predicting (through computation) a time varying concentration of the component of interest in the simulated in vivo conditions of the subject, the simulated plasma sample of the subject, and the simulated in vitro assays of the plasma sample of the subject.

For example, the simulated plasma sample of the subject can have mathematical expressions representing AT lowering, Factor VIII, and antithrombin concentrations. Likewise, the QSP model disclosed herein can predict, based on the mathematical expressions representing AT lowering, Factor VIII, and antithrombin concentrations in the simulated plasma sample, the outcome of a simulated thrombin generation assay (TGA), including peak thrombin, AUC, and lag time, or an activated partial thromboplastin time (aPTT) assay.

In some embodiments, the mathematical expressions are differential equations providing time-dependent representations of the species of interest in the simulated in vivo conditions of the subject, the simulated plasma sample of the subject, and the simulated in vitro assays of the plasma sample of the subject. The differential equations can include vectors and/or matrixes of rate constants or other parameters affecting the concentration or amount of the species of interest. In some embodiments, the individual differential equations and or other mathematical representations of the species of interest in the simulated in vivo conditions of the subject, the simulated plasma sample of the subject, and the simulated in vitro assays of the plasma sample of the subject are solved simultaneously, typically by numerical means, to provide time-dependent values of each of the species in the model.

To solve for the time-dependent concentrations of the species in the model, in addition to the rate constants and other information relating to the reaction between species and simulated assay outputs (e.g., via differential equations programmed into a computational system), a set of subject-specific parameters can be included in the model, e.g., for a simulated patient or a simulated patient population. These include intrinsic parameters and extrinsic parameters. Intrinsic parameters are parameters specific to the subject and outside the control of a physician or clinician treating the subject. Extrinsic parameters are parameters under the control of the physician or clinician. Examples of intrinsic parameters include the mass of the subject, and characteristics of the plasma sample that are specific to the subject. Examples of extrinsic parameters include the dose of one or more administered therapeutics (e.g., AT lowering or supplemental Factor VIII), frequency of dose of the one or more administered therapeutics, other medicaments administered concurrently with the one or more administered therapeutics, and the like.

Rate constants and other parameters programmed into the QSP model disclosed herein (e.g., included in ordinary differential equations for numerical solution) can be obtained from various sources including literature references, clinical data, and calibration by experimentation. Calibration can be conducted in vitro or in vivo.

### Context for Disclosed Computational Embodiments

Certain embodiments disclosed herein relate to systems for generating and/or using QSP models. Certain embodiments disclosed herein relate to methods for generating and/or using a QSP model implemented on such systems. A system for generating a QSP model can be configured to analyze data for calibrating the expressions or relationships used to represent hemostatic equivalency of fitusiran prophylaxis (i.e., AT lowering) in a subject. In such calibration, the system can determine rate constants or other parameter values charactering peak thrombin levels in the subject after treatment with fiusiran and/or supplemental Factor VIII. A system for generating a QSP model can also be configured to receive data and instructions such as program code representing physical processes in the plasma of the subject. In this manner, a QSP model is generated or programmed on such system. A programmed system for using a QSP model can be configured to (i) receive input such as pharmacological conditions characterizing a subject and (ii) execute instructions that determine the hemostatic equivalency of fitusiran prophylaxis (i.e., AT lowering) in a subject in the plasma of the subject. To this end, the system can calculate time-dependent concentrations of antithrombin in the plasma of the subject.

Many types of computing systems having any of various computer architectures can be employed as the disclosed systems for implementing QSP models and algorithms for generating and/or calibrating such models. For example, the systems can include software components executing on one or more general purpose processors or specially designed processors such as programmable logic devices (e.g., Field Programmable Gate Arrays (FPGAs)). Further, the systems can be implemented on a single device or distributed across multiple devices. The functions of the computational elements can be merged into one another or further split into multiple sub-modules.

In some embodiments, code executed during generation or execution of a QSP model on an appropriately programmed system can be embodied in the form of software elements which can be stored in a nonvolatile storage medium (such as optical disk, flash storage device, mobile hard disk, etc.), including a number of instructions for making a computer device (such as personal computers, servers, network equipment, etc.).

At one level a software element is implemented as a set of commands prepared by the programmer/developer. However, the module software that can be executed by the computer hardware is executable code committed to memory using “machine codes” selected from the specific machine language instruction set, or “native instructions,” designed into the hardware processor. The machine language instruction set, or native instruction set, is known to, and essentially built into, the hardware processor(s). This is the “language” by which the system and application software communicates with the hardware processors. Each native instruction is a discrete code that is recognized by the processing architecture and that can specify particular registers for arithmetic, addressing, or control functions; particular memory locations or offsets; and particular addressing modes used to interpret operands. More complex operations are built up by combining these simple native instructions, which are executed sequentially, or as otherwise directed by control flow instructions.

The inter-relationship between the executable software instructions and the hardware processor is structural. In other words, the instructions per se are a series of symbols or numeric values. They do not intrinsically convey any information. It is the processor, which by design was preconfigured to interpret the symbols/numeric values, which imparts meaning to the instructions.

The models used herein can be configured to execute on a single machine at a single location, on multiple machines at a single location, or on multiple machines at multiple locations. When multiple machines are employed, the individual machines can be tailored for their particular tasks. For example, operations requiring large blocks of code and/or significant processing capacity can be implemented on large and/or stationary machines. Such operations can be implemented on hardware remote from the site where a sample is acquired or where data is input; e.g., on a server or server farm connected by a network to a field device that captures the sample image. Less computationally intensive operations can be implemented on a portable or mobile device used on site for clinical evaluation.

In addition, certain embodiments relate to tangible and/or non-transitory computer readable media or computer program products that include program instructions and/or data (including data structures) for performing various computer-implemented operations. Examples of computer-readable media include, but are not limited to, semiconductor memory devices, phase-change devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media can be directly controlled by an end user or the media can be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that can be executed by the computer using an interpreter.

In various embodiments, the data or information employed in the disclosed methods and apparatus is provided in an electronic format. Such data or information can include pharmacological conditions associated with administering fitusiran and/or supplemental Factor VIII to a subject, intrinsic characteristics of a subject, model parameters such as rate constants, PK/PD results, and the like. As used herein, data or other information provided in electronic format is available for storage on a machine and transmission between machines. Conventionally, data in electronic format is provided digitally and can be stored as bits and/or bytes in various data structures, lists, databases, etc. The data can be embodied electronically, optically, etc.

In certain embodiments, a QSP model can each be viewed as a form of application software that interfaces with a user and with system software. System software typically interfaces with computer hardware and associated memory. In certain embodiments, the system software includes operating system software and/or firmware, as well as any middleware and drivers installed in the system. The system software provides basic non-task-specific functions of the computer. In contrast, the modules and other application software are used to accomplish specific tasks. Each native instruction for a module is stored in a memory device and is represented by a numeric value.

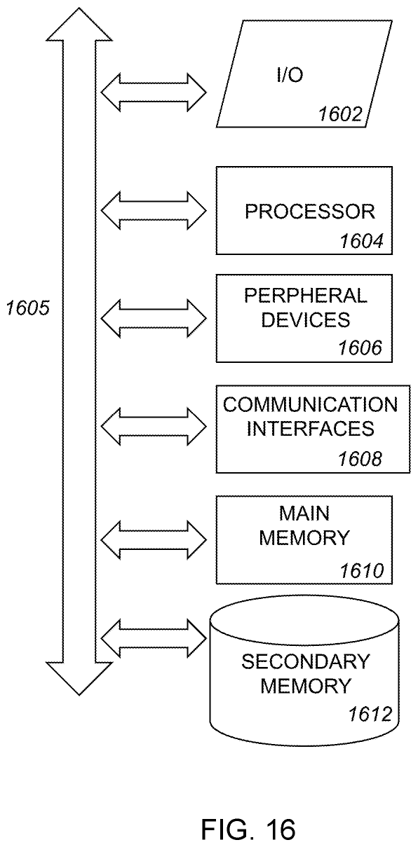

An example computer system 1600 is depicted in FIG. 16. As shown, computer system 1600 includes an input/output subsystem 1602, which can implement an interface for interacting with human users and/or other computer systems depending upon the application. Embodiments of the invention can be implemented in program code on system 1600 with I/O subsystem 1602 used to receive input program statements and/or data from a human user (e.g., via a GUI or keyboard) and to display them back to the user. The I/O subsystem 1602 can include, e.g., a keyboard, mouse, graphical user interface, touchscreen, or other interfaces for input, and, e.g., an LED or other flat screen display, or other interfaces for output.

Program code can be stored in non-transitory media such as persistent storage 1612 or memory 1612 or both. One or more processors 1604 reads program code from one or more non-transitory media and executes the code to enable the computer system to accomplish the methods performed by the embodiments herein, such as those involved with generating or using a QSP model as described herein. Those skilled in the art will understand that the processor can accept source code, such as statements for executing training and/or modelling operations, and interpret or compile the source code into machine code that is understandable at the hardware gate level of the processor. A bus 1605 couples the I/O subsystem 1602, the processor 1604, peripheral devices 1606, communications interfaces 1608, memory 1610, and persistent storage 1612.

### Methods of Treatment

The disclosure provides a method of treating HEMA and/or HEMB which includes administering one or more therapeutic compositions. In some embodiments, the therapeutic compositions include, for example, fitusiran and/or supplemental Factor VIII.

The disclosure also provides methods of administering an effective amount of fitusiran to the subject to achieve Factor VIII equivalency of about 10% to about 50%. As used herein, the term “Factor VIII equivalency” refers to the equivalent level of Factor VIII producing similar coagulation potential, as measured by thrombin generation assay metrics. A Factor VIII equivalency of 10-50% (e.g., 20-40%) is targeted to mitigate the risk of vascular thrombotic events while maintaining a favorable benefit-risk balance for patients on fitusiran. Thus, so long as the patient reaches this targeted Factor VIII equivalency, there is no need for the patient to receive a higher fitusiran dosage, more frequent dosing, or supplementary treatment.

In some embodiments, the disclosure features a method of reducing AT concentration in plasma of a subject including administering one or more therapeutic compositions as described herein, for example, fitusiran and/or supplemental Factor VIII.

In some embodiments, AT concentration in plasma of a subject is reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the AT concentration seen in the subject before administration of the composition.

In some embodiments, the disclosure features a method of increasing thrombin concentration in plasma of a subject including administering one or more therapeutic compositions as described herein, for example, fitusiran and/or supplemental Factor VIII.

In some embodiments, treatment according to the methods disclosed herein results in improvement, stabilization, or slowing of change in symptoms of HEMA or HEMB. In some embodiments, treatment according to the methods disclosed herein results in a reduction of annualized episodes of spontaneous or excessive bleeding by 10%, 20%, 30%, 40%, 50%, 60%, 70%, 80%, 90%, or more.

In some embodiments, efficacy of treatment is measured by improvement or slowing of progression in symptoms of HEMA or HEMB. In some embodiments, efficacy of treatment is measured by a decrease of excessive bleeding after trauma or surgery. In some embodiments, efficacy of treatment is measured by prevention of hematuria in a subject.

In some embodiments, efficacy of treatment is measured by laboratory findings including a reduced activated partial thromboplastin time (aPTT). In some embodiments, aPTT is reduced by 10%, 20%, 30%, 40%, 50%, 60%, 70%, 80%, 90%, or more. In some embodiments, efficacy of treatment is measured by laboratory findings including a reduced prothrombin time (PT). In some embodiments, PT is reduced by 10%, 20%, 30%, 40%, 50%, 60%, 70%, 80%, 90%, or more.

In some embodiments, the QSP model disclosed herein may be used to adjust the dosing regimen.

Evaluating the sensitivity of peak thrombin level to model parameters as disclosed herein can facilitate development of new treatment modalities which can target different aspects of hemostasis in patients with HEMA or HEMB. For example, the results of the AT lowering and peak thrombin predictions by the QSP model described herein may provide insight into the most effective combination of fitusiran and/or supplemental clotting factor for therapeutic intervention for patients with HEMA or HEMB. In some embodiments, the results of the AT lowering and peak thrombin predictions by the QSP model described herein may provide for selection of a dosing regimen for fitusiran and/or supplemental clotting factor for patients with HEMA or HEMB. In some embodiments, the results of the AT lowering and peak thrombin predictions by the QSP model described herein may provide for adjustment of a dosing regimen for fitusiran and/or supplemental clotting factor for patients with HEMA or HEMB.

Patients on fitusiran are monitored for hemostasis parameters, e.g., coagulation parameters (D-dimer, prothrombin fragment 1+2, and fibrinogen) and for signs and symptoms of vascular thrombotic events. Such signs and symptoms may include, but are not limited to, severe or persistent headache, headache with nausea and vomiting, chest pain and/or tightness, coughing up blood, trouble breathing, abdominal pain, fainting or loss of consciousness, swelling or pain in the arms or legs, vision problems, weakness and/or sensory deficits, and changes in speech. An evaluation of signs and symptoms potentially consistent with vascular thrombosis should include appropriate imaging studies as applicable. For the diagnosis of cerebral venous sinus thrombosis magnetic resonance imaging venogram (MRV) or computed tomography venogram (CTV) is recommended.

If a patient develops thrombosis while on fitusiran, AT reversal may be administered in combination with a replacement factor or BPA and appropriate anticoagulation. AT reversal should follow labeled product recommendations for the prevention of perioperative thrombosis in patients with AT deficiency, and individualize patient doses to target 80-120% AT activity. The use of plasma derived AT may be preferable to recombinant AT, given its longer half-life.

Bleeding events in patients on fitusiran may be managed by on-demand administration of a replacement factor (recombinant or plasma-derived Factor VIII or Factor IX) or a BPA (e.g., fresh-frozen plasma (FFP); rFVIIa; and aPCC). The amount of the factor or BPA must be reduced in patients on fitusiran to prevent vascular thrombosis. See, e.g., WO 2019/014187.

### EXAMPLES

The invention is further described in the following examples, which do not limit the scope of the invention described in the claims.

### Example 1: Thrombin Generation Assay (TGA) Simulations

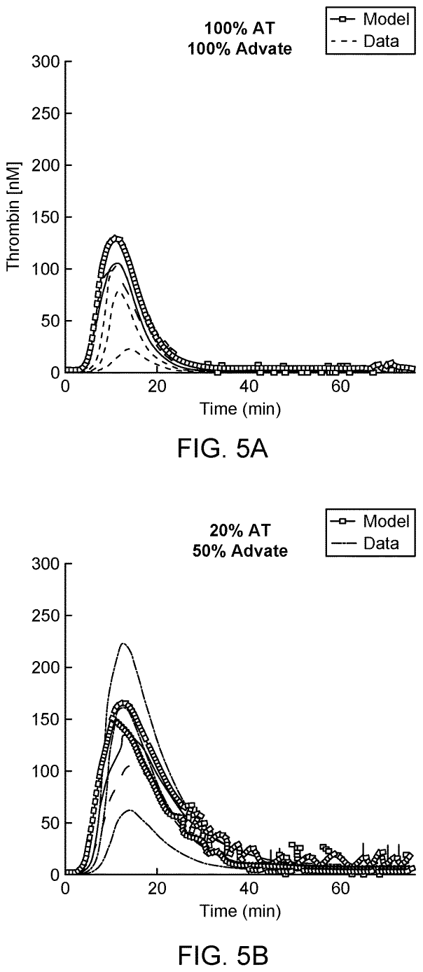

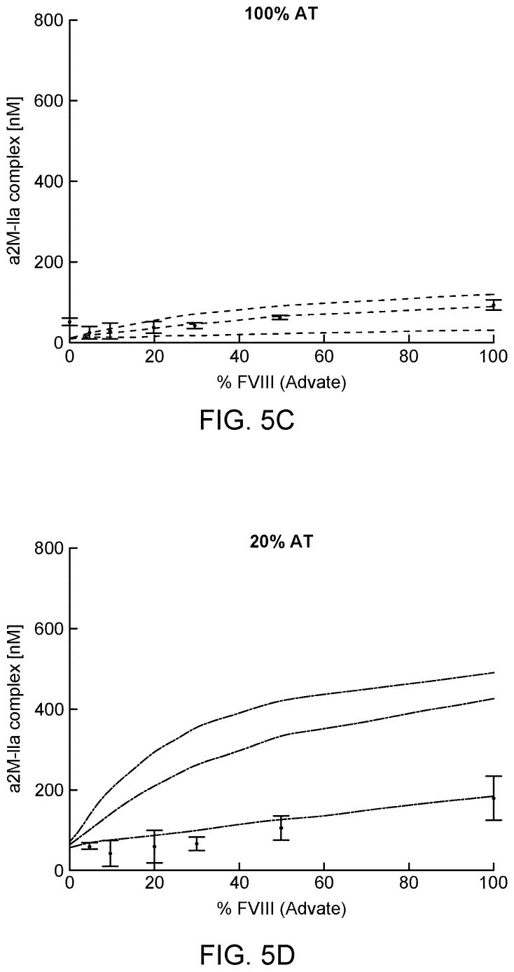

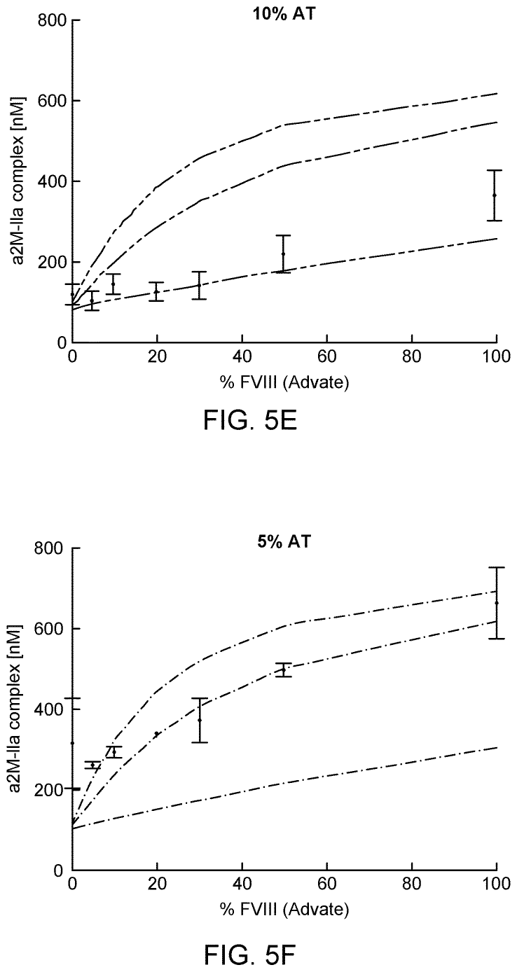

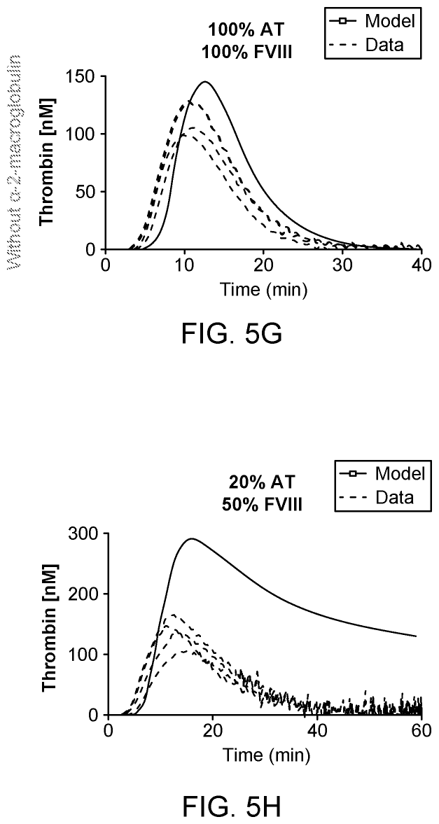

In order to validate the QSP models disclosed herein, predictions of peak thrombin (nM) based on the model were compared to thrombin generation assay (TGA) results from human plasma. TGA simulations were performed across different percentages of AT and FVIII (FIGS. 5A-5B). These results indicate that by including α-2-macroglobulin reactions, the QSP model disclosed herein accurately described characteristics of thrombin dynamics during the thrombin generation assay at different antithrombin levels. FIG. 5G and FIG. 5H are plots of thrombin concentration in plasma (nM) over time, comparing thrombin levels predicted by a previous model of the coagulation cascade that did not include α-2-macroglobulin (black line) to previously collected clinical data, with 100% antithrombin levels and 100% supplemental Factor VIII (Advate) levels (FIG. 5G) and 20% antithrombin levels and 50% supplemental Factor VIII (FIG. 5H). As shown by FIG. 5H, the model of the coagulation cascade that did not include α-2-macroglobulin (black line) did not accurately simulate thrombin generation at lower antithrombin levels (e.g., 20% AT).

FIGS. 5C-5F are plots of α2M-IIa complex concentration (y-axis) against supplemental Factor VIII (Advate) levels (x-axis), comparing α2M-IIa complex levels predicted by the QSP model disclosed herein (lines of the plots) to previously collected clinical data (datapoints with error bars), with 100% AT (FIG. 5C), 20% AT (FIG. 5D), 10% AT (FIG. 5E), and 5% AT (FIG. 5F). These results indicate that the QSP model also accurately represents the formation of the complex between thrombin and α-2-macroglobulin at different levels of antithrombin and factor VIII.

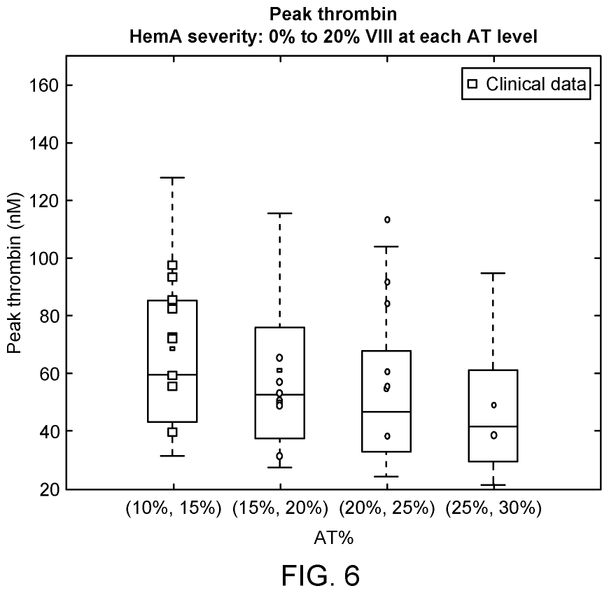

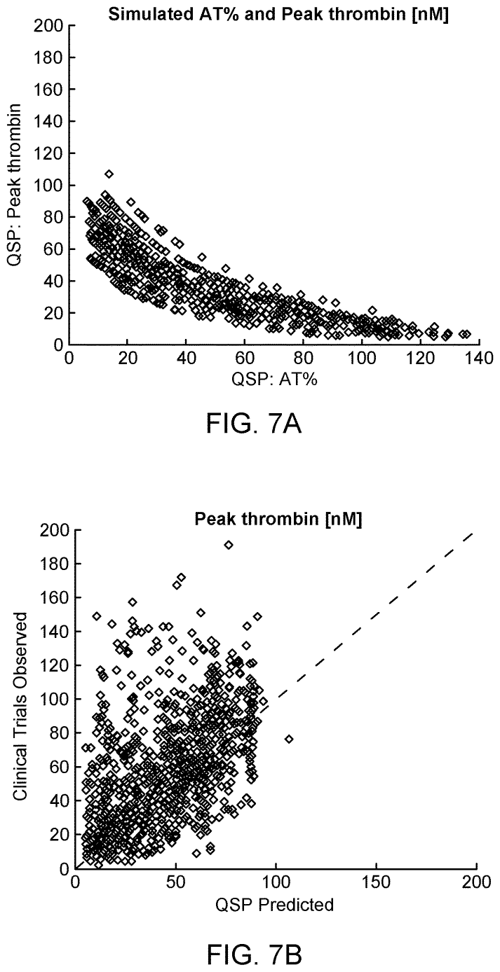

FIG. 6 shows peak thrombin levels (nM) for patients with a range of HEMA severity (residual factor VIII levels) observed in clinical data at different AT % ranges (x-axis). FIGS. 7A-7B represent aggregate predictive performance of the QSP model.

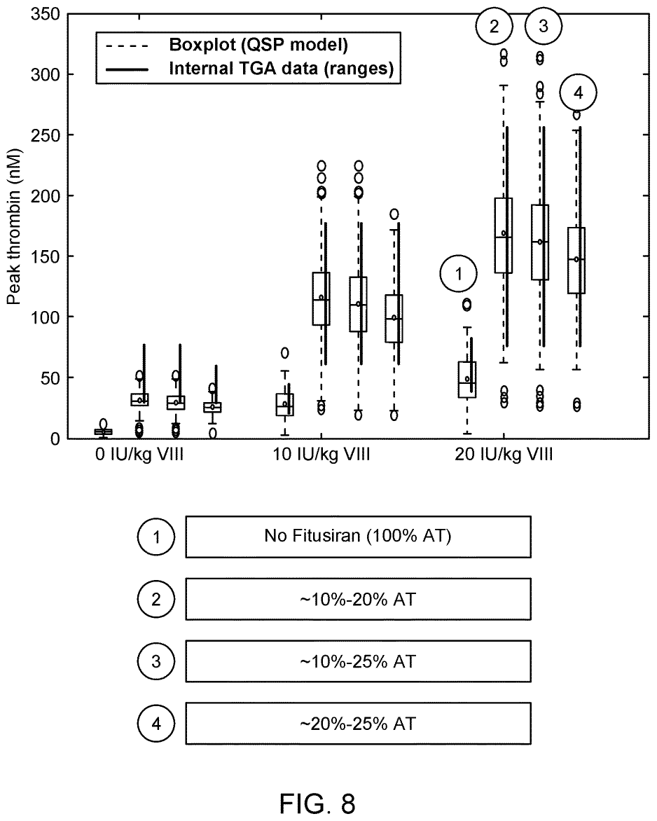

These simulations reproduced experimental TGA data from AT- and FVIII-double depleted plasma that was spiked with different levels of AT and/or FVIII. For these in vitro experiments, Factor VIII (FVIII) and AT-double depleted plasma was generated from congenital FVIII-deficient plasma by immunodepleting AT using an anti-AT antibody and recombinant AT protein was spiked back in to achieve different levels of AT. Factor VIII was spiked in to replicate dosage of 0 IU/kg, 10 IU/kg, and 20 IU/kg supplemental Factor VIII. AT was spiked in at prescribed amounts corresponding to 100% AT, 10-20% AT, 10-25% AT and 20-25% AT. TGA was performed on spiked-in plasma samples. As shown in FIG. 8, predictions of the QSP model (boxplot) are concordant with TGA data (ranges) for the three Factor VIII dosages and for each of the four ranges of AT % at each Factor VIII dose.

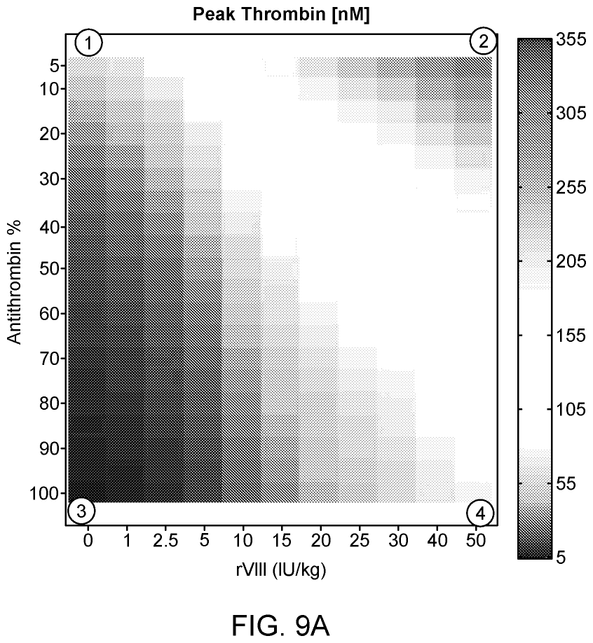

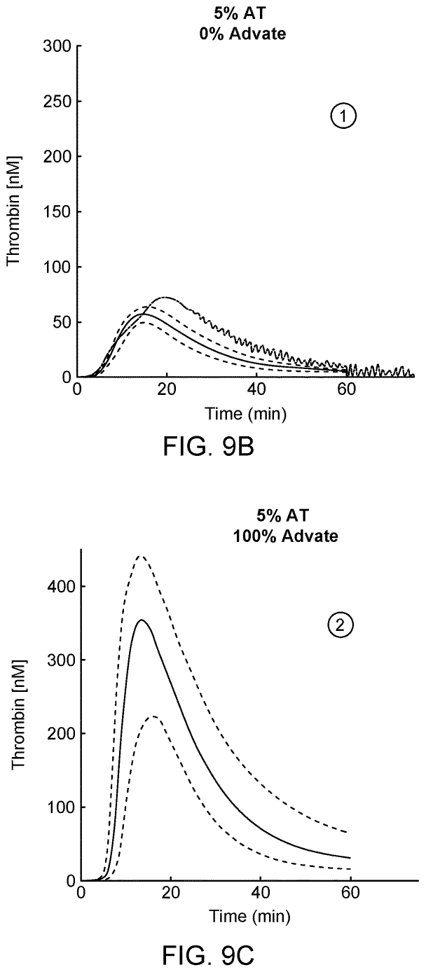

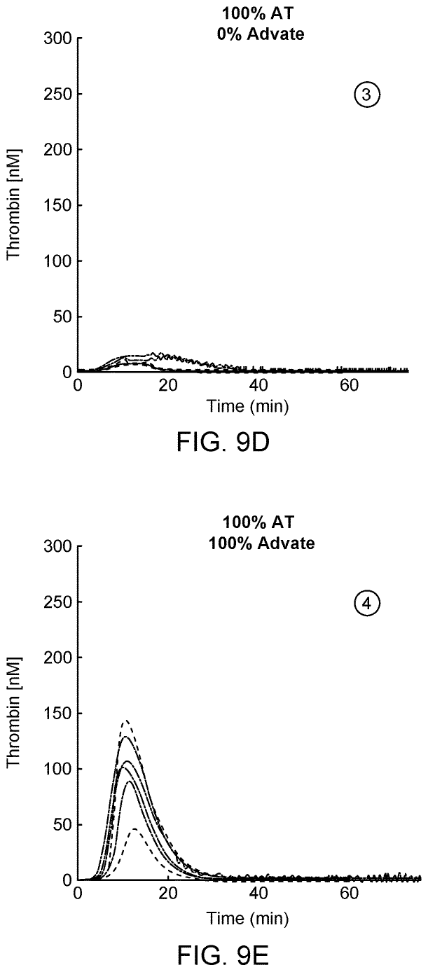

Next, predictions of the model were compared to spike-in TGA results on human plasma for a wider range of AT percentages and Factor VIII dosages. FIG. 9A depicts a heatmap of predicted peak thrombin (nM) for a range of AT % (y-axis) and Factor VIII dosages (x-axis). Labels “1,” “2,” “3,” and “4,” correspond to relative extremes for ranges of AT % and supplemental Factor VIII dosage. The QSP model was used to predict peak thrombin for these ranges, and TGA was performed on AT-depleted plasma for these ranges. FIG. 9B shows predicted and laboratory spike-in results for 5% AT and 0% supplemental Factor VIII. FIG. 9C shows predicted and laboratory spike-in results for 5% AT and 100% supplemental Factor VIII. FIG. 9D shows predicted and laboratory spike-in results for 100% AT and 0% supplemental Factor VIII. FIG. 9E shows predicted and laboratory spike-in results for 100% AT and 100% supplemental Factor VIII. For each condition, the QSP model predicted results correspond to the laboratory TGA results, indicating that the QSP model accurately predicts peak thrombin (nM) over a wide range of conditions.

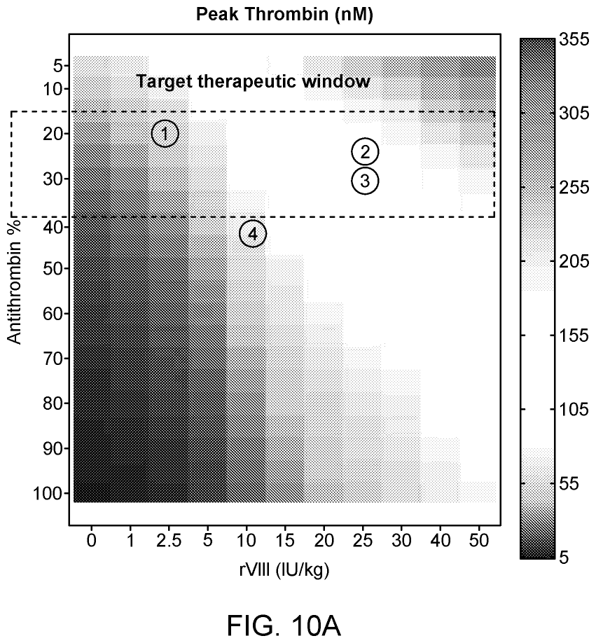

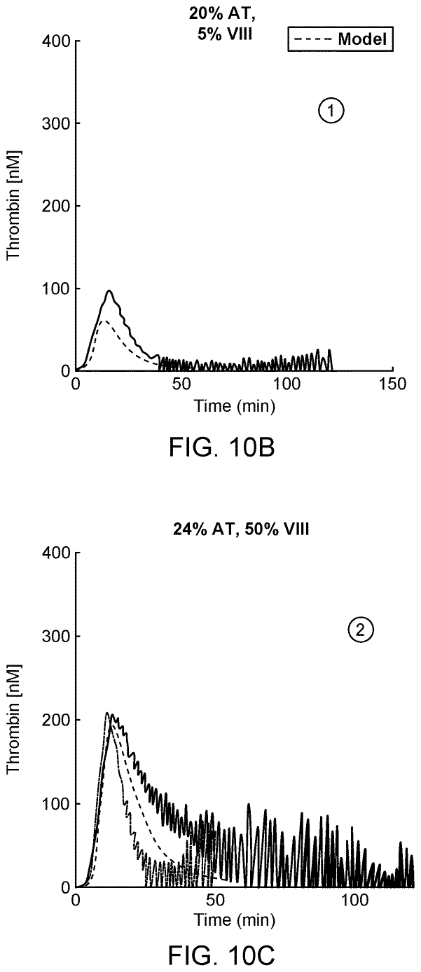

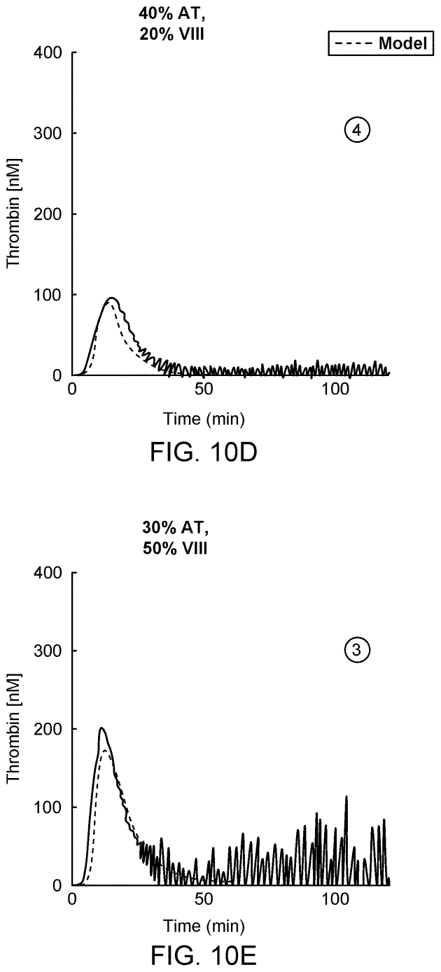

Next, predictions of the model were compared to spike-in TGA results on human plasma for intermediate ranges of AT percentages and Factor VIII dosages. FIG. 10A depicts a heatmap of predicted peak thrombin (nM) for a range of AT % (y-axis) and Factor VIII dosages (x-axis). Labels “1,” “2,” “3,” and “4,” correspond to intermediate ranges of AT % and supplemental Factor VIII dosage that overlap the target therapeutic window for clinical treatment of HEMA and HEMB patients. The QSP model was used to predict peak thrombin for these ranges, and TGA was performed on AT-depleted plasma for these ranges. FIG. 10B shows predicted and laboratory spike-in results for 20% AT and 5% supplemental Factor VIII. FIG. 10C shows predicted and laboratory spike-in results for 24% AT and 50% supplemental Factor VIII. FIG. 10D shows predicted and laboratory spike-in results for 40% AT and 20% supplemental Factor VIII. FIG. 10E shows predicted and laboratory spike-in results for 30% AT and 50% supplemental Factor VIII. For each condition, the QSP model predicted results are in adequate agreement to the laboratory TGA results, indicating that the QSP model accurately predicts peak thrombin (nM) over intermediate ranges of AT % and supplemental Factor VIII dosage that overlap the target therapeutic window for clinical treatment of HEMA and HEMB patients.

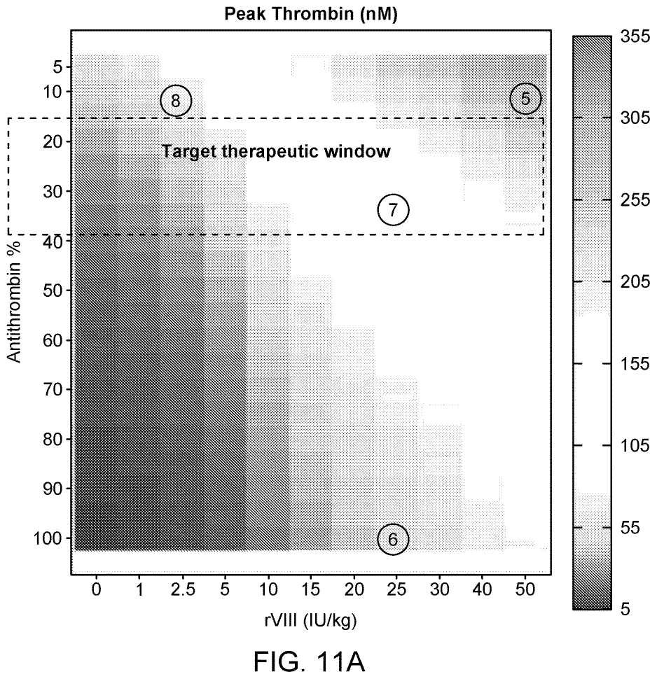

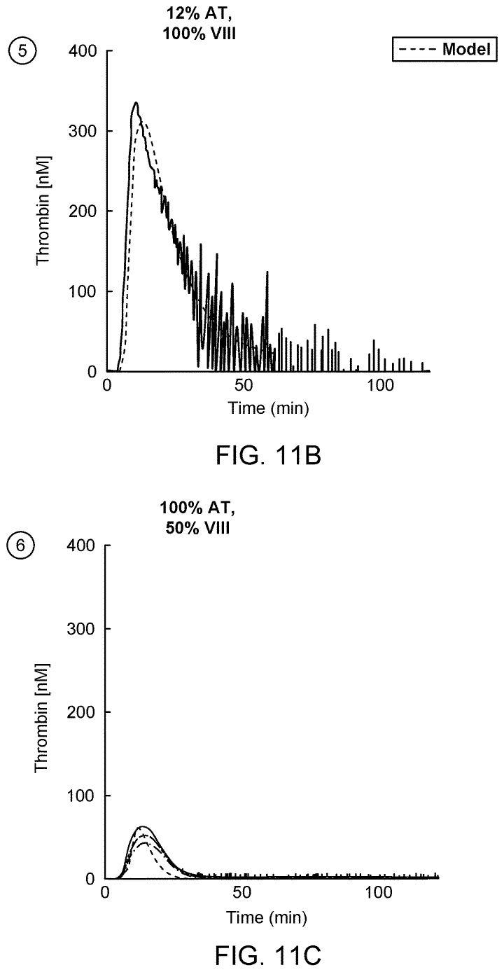

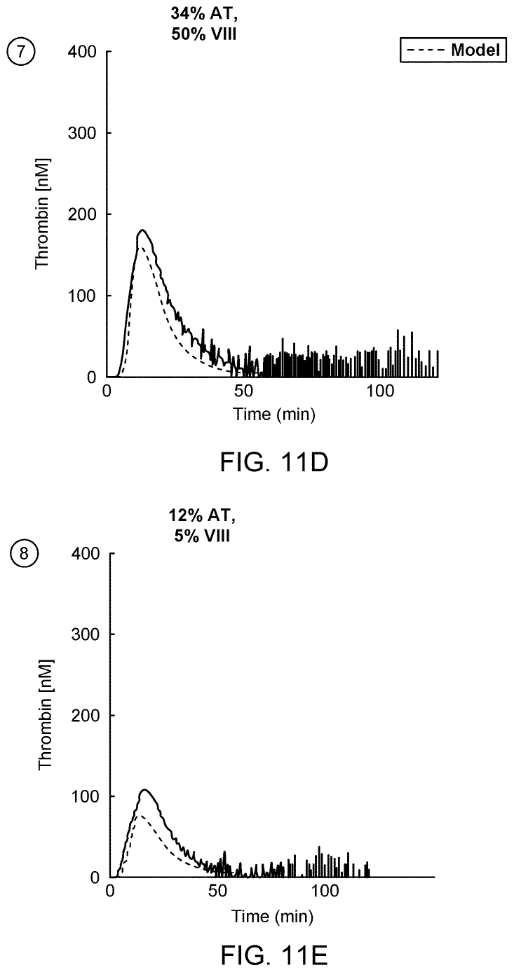

Next, predictions of the model were compared to spike-in TGA results on human plasma for additional ranges of AT percentages and Factor VIII dosages. FIG. 11A depicts a heatmap of predicted peak thrombin (nM) for a range of AT % (y-axis) and Factor VIII dosages (x-axis). Labels “5,” “6,” “7,” and “8,” correspond to additional ranges of AT % and supplemental Factor VIII dosage, some of which overlap the target therapeutic window for clinical treatment of HEMA and HEMB patients. The QSP model was used to predict peak thrombin for these ranges, and TGA was performed on AT-depleted plasma for these ranges. FIG. 11B shows predicted and laboratory spike-in results for 12% AT and 100% supplemental Factor VIII. FIG. 11C shows predicted and laboratory spike-in results for 100% AT and 50% supplemental Factor VIII. FIG. 11D shows predicted and laboratory spike-in results for 34% AT and 50% supplemental Factor VIII. FIG. 11E shows predicted and laboratory spike-in results for 12% AT and 5% supplemental Factor VIII. For each condition, the QSP model predicted results correspond to the laboratory TGA results, indicating that the QSP model accurately predicts peak thrombin (nM) over intermediate ranges of AT % and supplemental Factor VIII dosage.

### Example 2: Activated Partial Thromboplastin Time (aPTT) Assay Simulations

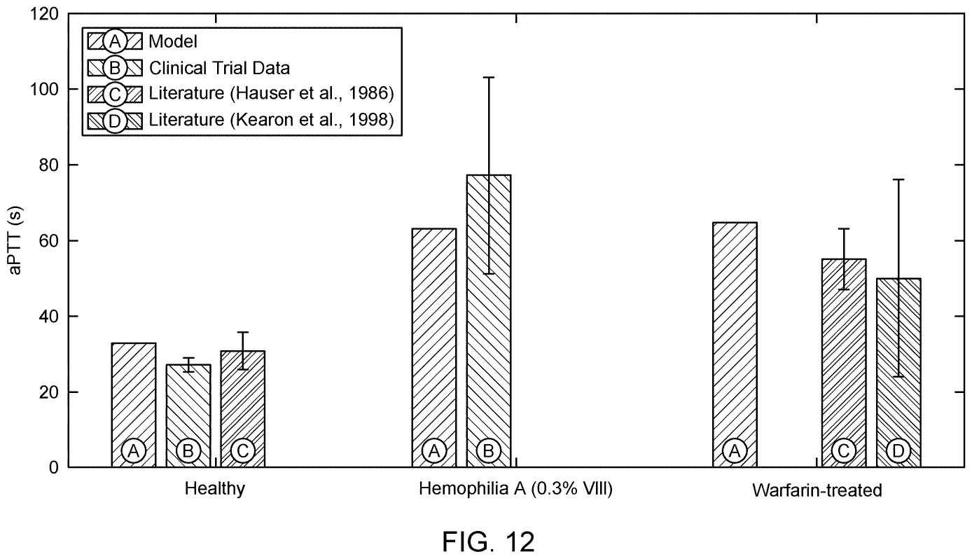

In order to further validate the QSP models disclosed herein, predictions of aPTT results based on the model were compared to aPTT results from clinical trial data and from result previously reported in the literature. As shown in FIG. 12, aPTT results were predicted for healthy individuals, patients with HEMA, and non-HEMA patients who were treated with Warfarin for other bleeding disorders as a validation exercise. The QSP model adequately predicted aPTT results for these three groups, with results corresponding to clinical trial data and previously reported data from the literature. The results indicate that the QSP model accurately describes multiple aspects of the coagulation pathway.

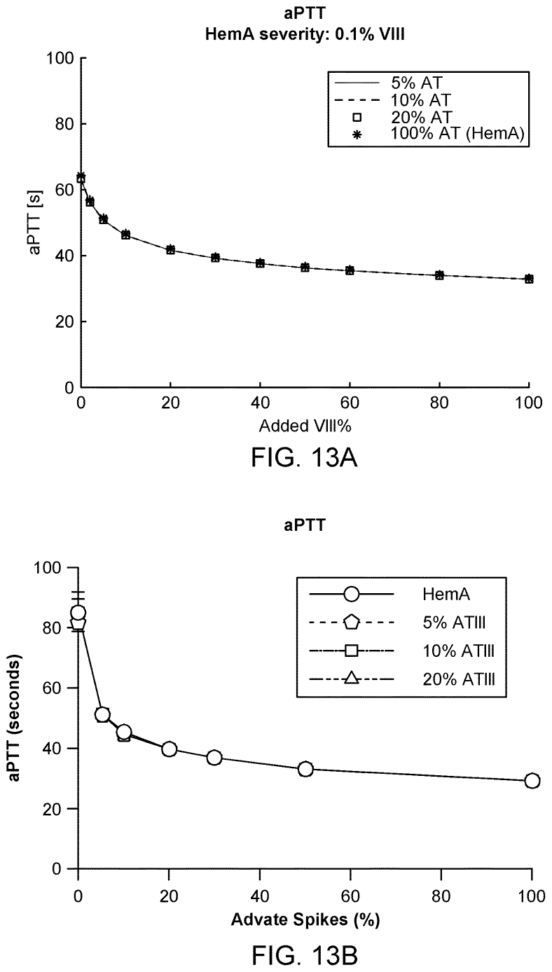

Next, aPTT results were predicted over a range of AT percentages and supplemental Factor VIII percentages, and predictions of the QSP model were compared to laboratory aPTT results for AT- and Factor VIII-double depleted plasma that was spiked with different levels of AT and/or Factor VIII as described in Example 1. FIG. 13A shows results for the predictions of the QSP mode and FIG. 13B shows laboratory aPTT results for the spiked-in plasma. These results indicate that the QSP model adequately predicts aPTT results over a wide range of AT percentages and supplemental Factor VIII percentages.

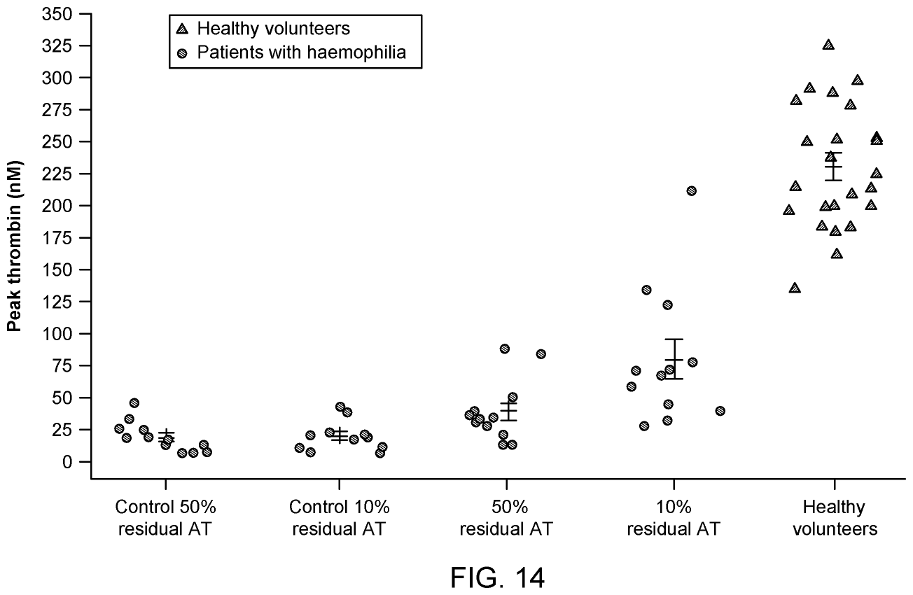

Finally, QSP model-predicted effects of AT reduction on peak thrombin according to the QSP model disclosed herein were compared to results reported by Livnat et al. (Livnat T, et al. Thrombin generation in plasma of patients with haemophilia A and B with inhibitors: Effects of bypassing agents and antithrombin reduction. Blood Cells Mol Dis. 2020 May;82:102416.). FIG. 14 is a plot reproduced from that published report, showing peak thrombin (nM) for control 50% residual AT, control 10% residual AT, 50% residual AT, 10% residual AT, and healthy volunteers. For patients with 10% residual AT, Livnat et al. reported ˜35% peak thrombin (nM) relative to healthy volunteers, and the QSP model predicted 30-45% peak thrombin (nM) relative to healthy volunteers. For patients with 50% residual AT, Livnat et al. reported ˜17% peak thrombin (nM) relative to healthy volunteers, and the QSP model predicted 10-16% peak thrombin (nM) relative to healthy volunteers.

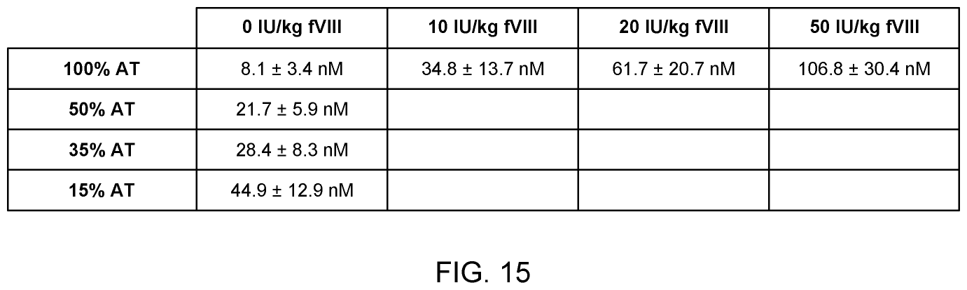

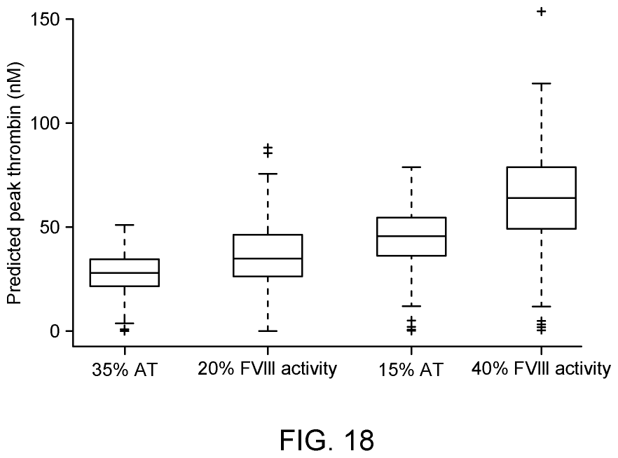

A simulated virtual population (n=1000) of severe Hemophilia A (0.1% residual Factor VIII) was also tested. The virtual population was generated based on calibration of the QSP model to individual patient pharmacokinetic, antithrombin, and thrombin generation assay data from completed fitusiran clinical studies. The virtual population was applied to simulate peak thrombin associated with AT lowering or associated with supplemental factor VIII. FIG. 15 show peak thrombin (mean±standard deviation) in the simulated virtual population. FIG. 18 is a plot of predicted peak thrombin (nM) based on results of the simulated virtual population generated based on calibration of the QSP model from patient data from clinical studies. The plot shows predicted peak thrombin (nM) for therapeutic antithrombin levels of 15% and 35% with fitusiran and FVIII activity of 20% and 40% in people with hemophilia A. The target therapeutic antithrombin range of fitusiran prophylaxis treatment at steady-state is 15-35%. Based on this modelling analysis and the results of the simulated virtual population presented in FIG. 18, the peak thrombin generated at 15-35% antithrombin was comparable to peak thrombin observed at 20-40% of factor VIII activity in people with hemophilia A. These results indicate that the QSP model disclosed herein predicts peak thrombin levels (nM) that are concordant with previously published clinical data from the literature.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of preparing a quantitative systems pharmacology model for predicting thrombin levels in plasma of a subject, the method comprising:
(a) providing a plurality of relationships and/or parameters characterizing time-dependent antithrombin (AT) levels in plasma of a subject in response to an agent targeting antithrombin in the plasma of the subject;
(b) providing a plurality of relationships and/or parameters characterizing α-2-macroglobulin levels in the plasma of the subject;
(c) determining a plurality of rate constants for the plurality of relationships and/or parameters characterizing time-dependent AT levels in plasma of a subject in response to an agent targeting antithrombin in the plasma of the subject;
(d) determining a rate constant for the relationship or the parameter characterizing α-2-macroglobulin levels in the plasma of the subject; and
(e) programming a computational system with (i) the plurality of rate constants for the plurality of relationships and/or parameters characterizing time-dependent AT levels in plasma of a subject in response to an agent targeting antithrombin in the plasma of the subject; and (ii) the rate constant for the relationship or the parameter characterizing α-2-macroglobulin levels in the plasma of the subject,
whereby the computational system is programmed to (i) solve a system of expressions under a defined set of pharmacological conditions, wherein the system of expressions comprises the plurality of relationships and/or parameters characterizing time-dependent AT levels in plasma of a subject in response to an agent targeting antithrombin in the plasma of the subject and the plurality of relationships and/or parameters characterizing α-2-macroglobulin levels in the plasma of the subject and (ii) output thrombin levels in plasma of a subject after administration of the agent targeting antithrombin in the plasma of the subject under the defined set of pharmacological conditions.

2. The method of claim 1, where the agent targeting antithrombin in the plasma of the subject is an siRNA therapeutic.

3. The method of claim 2, wherein the siRNA therapeutic is fitusiran.

4. The method of any one of claims 1-3, wherein AT levels are below 35%.

5. The method of any one of claims 1-4, wherein the method calculates the formation and the degradation of α-2-macroglobulin-thrombin complex.

6. The method of any one of claims 1-5, wherein α-2-macroglobulin has a concentration of about 3 to about 6 μM.

7. A computer-implemented method for modeling and simulating thrombin levels in the plasma of a subject, the method comprising:
obtaining a quantitative systems pharmacology (QSP) model of thrombin levels in the plasma of a subject, wherein the QSP model is configured to represent results of a thrombin generation assay (TGA) in response to plasma levels of antithrombin (AT), α-2-macroglobulin, and a pharmaceutical agent targeting antithrombin in the plasma of the subject;
determining parameters affecting peak thrombin as indicated by the TGA;
assigning the parameters affecting peak thrombin to a virtual patient population; and
processing the virtual patient population using the QSP model to provide processed data, wherein the processed data comprises an amount of the agent targeting antithrombin in the plasma of the subject.

8. The computer-implemented method of claim 7, wherein the plasma levels of AT are below 35%.

9. The computer-implemented method of claim 7 or 8, wherein the method calculates the formation and the degradation of α-2-macroglobulin-thrombin complex.

10. The computer-implemented method of any one of claims 7-9, further comprising displaying the processed data.

11. The computer-implemented method of any one of claims 7-10, further comprising:
determining pharmacokinetic parameters of the agent targeting antithrombin in the plasma of the subject;
determining pharmacokinetic parameters of one or more additional therapeutics; and
processing the pharmacokinetic parameters of the agent targeting antithrombin in the plasma of the subject and the pharmacokinetic parameters of the one or more additional therapeutics to determine effectiveness of the combination of the agent targeting antithrombin in the plasma of the subject and one or more additional therapeutics.

12. The computer-implemented method of any one of claims 7-11, wherein the agent targeting antithrombin in the plasma of the subject is an RNAi therapeutic.

13. The method of claim 12, wherein the RNAi therapeutic is an siRNA therapeutic.

14. The method of claim 12, wherein the siRNA therapeutic is fitusiran.

15. A computer-implemented method for determining Factor VIII equivalency in a subject, the method comprising:
obtaining a quantitative systems pharmacology (QSP) model of thrombin levels in the plasma of a subject, wherein the QSP model is configured to represent the interaction of plasma levels of antithrombin (AT), α-2-macroglobulin, and thrombin;
determining parameters affecting a thrombin generation assay; and
processing the QSP model to provide processed data, wherein the processed data indicates Factor VIII equivalency in a subject.

16. The computer-implemented method of claim 15, wherein the plasma levels of antithrombin is a time-dependent variable.

17. The method of claim 15 or 16, wherein the plasma levels of antithrombin are below 35%.

18. The computer-implemented method of any one of claims 15-17, wherein the formation and the degradation of thrombin-antithrombin (T-AT) are calculated.

19. The computer-implemented method of any one of claims 15-18, wherein the formation and the degradation of α-2-macroglobulin-thrombin complex are calculated.

20. The computer-implemented method of any one of claims 15-19, wherein the results of a thrombin generation assay (TGA) in response to the interaction of plasma levels of antithrombin (AT), α-2-macroglobulin, and thrombin are modeled.

21. The computer-implemented method of any one of claims 15-19, wherein peak thrombin as indicated by the TGA is used to determine Factor VIII equivalency.

22. The computer-implemented method of any one of claims 15-21, wherein the subject is administered an siRNA therapeutic.

23. The method of claim 22, wherein the siRNA therapeutic is fitusiran.

24. A method of achieving a Factor VIII equivalency of about 10% to about 50% in a hemophilia patient in need thereof, the method comprising
administering a prophylactically effective amount of fitusiran subcutaneously to the patient to achieve an AT level of 10-35% in the patient.

25. The method of claim 24, wherein the patient is a hemophilia A or hemophilia B patient.

26. The method of claim 25, wherein the patient is a hemophilia A patient with or without inhibitors, or a hemophilia B patient with or without inhibitors.

27. The method of any one of claims 24-26, wherein the method comprises achieving a Factor VIII equivalency of about 20% to about 40%.

28. The method of any one of claims 24-27, wherein the prophylactically effective amount of fitusiran is selected from about 1.25 mg, about 2.5 mg, about 5 mg, about 25 mg, about 30 mg, about 50 mg, and about 80 mg.

29. The method of any one of claims 24-28, wherein the prophylactically effective amount of fitusiran is administered about every month (or about every four weeks) or about once every two months (or about every eight weeks).

30. The method of any one of claims 24-29, wherein the prophylactically effective amount of fitusiran is about 50 mg administered about every month (or about every four weeks) or about once every two months (or about every eight weeks).

31. The method of any one of claims 24-29, wherein the prophylactically effective amount of fitusiran is about 20 mg administered about every month (or about every four weeks) or about once every two months (or about every eight weeks).

32. A method of treating hemophilia in a patient with or without inhibitors, the method comprising:
(a) providing a time-dependent antithrombin level in plasma of the patient;
(b) providing a plurality of relationships and/or parameters characterizing α-2-macroglobulin level in the plasma of the patient;
(c) programming a computational system with one or more rate constants for the relationship or the parameters characterizing thrombin level;
(d) determining hemostatic equivalency with Factor VIII in the patient; and
(e) administering to the patient a therapeutic agent.

33. The method of claim 32, wherein the therapeutic agent is Supplemental Factor VIII.

34. The method of claim 32, wherein the therapeutic agent is fitusiran.

35. The method of any one of claims 32-34, wherein hemostatic equivalency with Factor VIII is determined by modeling the interaction of plasma levels of antithrombin (AT), α-2-macroglobulin, and thrombin.

36. The method of any one of claims 32-35, wherein the time-dependent antithrombin level is below 35%.

37. A method of determining hemostatic equivalency with Factor VIII in a subject receiving treatment for hemophilia A or B, the method comprising
(a) providing a time-dependent antithrombin level in plasma of the subject;
(b) providing a plurality of relationships and/or parameters characterizing α-2-macroglobulin level in the plasma of the subject;
(c) programming a computational system with one or more rate constants for the relationship or the parameters characterizing thrombin level; and
(d) determining hemostatic equivalency with Factor VIII in the subject.

38. The method of claim 37, wherein the method further comprises:
administering supplemental Factor VIII to the subject.

39. The method of claim 37, wherein the method further comprises:
adjusting the dosage of fitusiran for the subject.
